(19) United States
(12) Patent Application Publication
(10) Pub. No.: US 2026/0345695 A1
(43) Pub. Date: Nov. 5, 2026

(54) COMPOSITION AND METHOD FOR DELIVERING A DRUG ACROSS THE BLOOD-BRAIN BARRIER USING BACTERIAL EXTRACELLULAR VESICLES

(71) Applicant: LISCURE BIOSCIENCES CO., LTD., Seongnam-si (KR)

(72) Inventor: Hwa Sup CHIN, Yongin-si (KR)

(73) Assignee: LISCURE BIOSCIENCES CO., LTD., Seongnam-si (KR)

(21) Appl. No.: 19/738,219

(22) Filed: Jul. 10, 2026

Related U.S. Application Data
(63) Continuation-in-part of application No. 18/015,783, filed on Jul. 13, 2021, now Pat. No. 12,697,357.

(30) Foreign Application Priority Data
Jul. 14, 2020 (KR) 10-2020-0087033

(51) Int. Cl. A61K 35/744; A61K 9/51; A61P 25/00; A61P 25/16; A61P 25/28; A61P 29/00; C12N 15/113; C12N 15/88

(52) U.S. Cl. CPC A61K 35/744; A61K 9/5184; A61P 25/00; A61P 25/16; A61P 25/28; A61P 29/00; C12N 15/113; C12N 15/88; C12N 2310/11; C12N 2320/32

(57) ABSTRACT

Extracellular vesicles derived from a strain, loaded with a drug, are disclosed. The extracellular vesicles are capable of crossing the blood-brain barrier (BBB), and thus can deliver the drug to the whole brain, including deep regions thereof, in vivo by mere intravenous administration. In addition, since the drug can be efficiently loaded onto the extracellular vesicles even under simple strain culture conditions, the extracellular vesicles can replace conventional invasive direct intracerebral drug injection methods, such as intracerebral injection, intracerebroventricular injection (ICV) and stereotaxic injection, and can thus be utilized as a platform technology effectively applicable to improving patient compliance and establishing long-term treatment strategies for CNS diseases and the like.

### CROSS REFERENCE TO RELATED APPLICATIONS

This application is a Continuation-in-Part of U.S. application Ser. No. 18/015,783 filed Jan. 12, 2023, which is a National Stage of International Application No. PCT/KR2021/009001, filed Jul. 13, 2021, claiming priority to Korean Patent Application No. 10-2020-0087033, filed Jul. 14, 2020.

### INCORPORATION OF SEQUENCE LISTING

The content of the electronically submitted sequence listing, file name: Q323414SEQLIST.xml; size: 6,135 bytes; and date of creation: Jul. 9, 2026, filed herewith, is incorporated herein by reference in its entirety.

### TECHNICAL FIELD

The present disclosure relates to a composition for preventing, ameliorating or treating a CNS disease, which contains Pediococcus inopinatus, a culture, a lysate, an extract or a fermented product thereof, or extracellular vesicles isolated therefrom as an active ingredient. The present disclosure further relates to a composition for drug delivery and a method for delivering a drug across the blood-brain barrier (BBB) into the brain.

### BACKGROUND ART

Central nervous system (CNS) diseases encompass a broad spectrum of diseases, disorders, and conditions involving structural or functional abnormality of the brain or central nervous system, including cerebrovascular diseases, degenerative brain diseases, neurogenetic diseases, inflammatory brain diseases, epilepsy, CNS tumors, and other CNS-related disorders. With the rapid global population aging, the incidence of CNS diseases has been increasing rapidly worldwide, placing a substantial burden on patients, caregivers, and healthcare systems.

Among CNS diseases, degenerative brain diseases refer to progressive degenerative brain diseases occurring due to selective death of neurons in specific parts of the brain because of various causes. Examples of the diseases include Alzheimer's disease, vascular dementia, Parkinson's disease, Huntington's disease, multiple sclerosis, Creutzfeldt-Jakob disease, Pick's disease and other diseases causing symptoms of dementia, such as depression, normal pressure hydrocephalus, metabolic brain disease, brain tumor, head injury, cognitive impairment and delirium.

Abnormal behaviors and disorders such as progressive cognitive dysfunction, progressive ataxia, muscle weakness, muscular atrophy, etc. occur due to causes not known thus with 1) gradual loss of the functions of specific cells in the brain and spinal cord, 2) decrease in the number of brain cells due to apoptosis, 3) death of neurons involved in the transport of neurotransmitters, 4) problem in signaling between nerves and formation or function of neural neurites and synapses and 5) reduced electrical activity of cranial nerve. In particular, Parkinson's disease is a disease wherein motor disorder, etc. occur due to the functional loss and decrease of dopamine neurons in the substantia nigra of the midbrain. Dopamine neurons are very vulnerable to oxidative stress because they form oxides through oxidative metabolism.

With the rapid global population aging, the patients with degenerative brain diseases are increasing rapidly. The Enforcement Decree of the Long-term Care Insurance Act classifies senile disease as follows according to Korean Standard Classification of Diseases: 1) Alzheimer's dementia, 2) vascular dementia, 3) dementia in other diseases classified elsewhere, 4) unspecified dementia, 5) Alzheimer's disease, 6) subarachnoid hemorrhage, 7) intracerebral hemorrhage, 8) other nontraumatic intracranial hemorrhage, 9) cerebral infarction, 10) stroke not specified as hemorrhage or infarction, 11) occlusion and stenosis of precerebral arteries, not resulting in cerebral infarction, 12) occlusion and stenosis of cerebral arteries, not resulting in cerebral infarction, 13) other cerebrovascular diseases, 14) cerebrovascular disorders in disease classified elsewhere, 15) sequelae of cerebrovascular diseases, 16) Parkinson's disease, 17) secondary parkinsonism, 18) parkinsonism in disease classified elsewhere, 19) other degenerative diseases of basal ganglia, 20) sequelae of palsy, and 21) tremor (involuntary, somewhat rhythmic, trembling of body parts).

Alzheimer's dementia is a chronic disease caused by degeneration of brain cells and gradual decline in cognitive function including memory and is characterized by gradual and fatal decline in cognitive function such as memory/judgment/language skill, etc., daily activities, personality, behavioral disorder, etc. Damage to brain cells due to excessive accumulation of amyloid beta, neurofibrillary tangles caused by hyperphosphorylation of tau protein, inflammatory response, oxidative damage, etc. is reported as its cause.

Vascular dementia refers to damage to brain tissue caused by cerebrovascular problems. Blockage or narrowing of cerebral vessels due to insufficient oxygen supply is reported as its cause.

Stroke includes ischemic stroke and hemorrhagic stroke. Ischemic stroke is caused by the partial damage of the brain caused by the blockage of cerebral vessels, and hemorrhagic stroke is caused by the rupture of cerebral vessels and pooling of blood in the affected area.

Microglia are present in the central nervous system (CNS) and are involved in the primary protection, phagocytosis and restoration of neurons. However, it is reported that activated microglia induce neuroinflammatory response. If the response persists, it can cause various neurodegenerative diseases.

It is reported that microglia activated by brain damage, brain cell death, neurotoxins, oxidative stress, etc. are involved in the pathomechanism of degenerative neurodegenerative diseases by nitric oxide (NO), reactive oxygen species (ROS), inflammatory cytokines and inflammatory enzymes. Therefore, it is expected that the amelioration of neuroinflammation through inhibition and regulation of microglial activation can play an important role in the prevention of degenerative neurodegenerative diseases. Reactive oxygen species (ROS) cause various diseases, and the overproduction/overexpression of reactive oxygen species leads to oxidative stress, cellular dysfunction and, ultimately, cell death or necrocytosis.

Under this background, prevention and treatment of CNS diseases using lactic acid bacteria are being researched recently, but there is no noteworthy result yet.

With the global increase in both aging-associated neurodegenerative disease and inherited neurological disorders, the demand of effective therapeutic strategies targeting diseases of the central nervous system (CNS) has continued to grow.

Accordingly, the development of nucleic acid therapeutics, including antisense oligonucleotides (ASOs) and short interfering RNAs (siRNAs), has attracted considerable attention, which can fundamentally suppress the cause of disease at the genetic level, has been receiving significant attention. However, despite their excellent target selectivity and therapeutic efficacy, such nucleic acid drugs face substantial obstacles to actual clinical application due to the particular characteristics of brain tissue. The brain is equipped with a defense system called the blood-brain barrier (BBB), in which vascular endothelial cells form highly elaborate tight junctions to block the entry of harmful substances. As a result, most nucleic acid drugs, which have a relatively large molecular weight, are hydrophilic, and carry a strong negative charge, are unable to cross the BBB on their own and migrate into brain tissue, which constitutes the most decisive technical barrier in the development of nucleic acid drug-based therapeutics for CNS disease.

In order to overcome the limitations of the BBB and efficiently deliver drugs into the brain, research on various drug delivery systems (DDS) has been actively conducted. Conventionally, attempts have been made to encapsulate drugs using carriers such as liposomes, polymer micelles, and synthetic nanoparticles, or to enhance permeation efficiency by modifying the surface with ligands that target receptors present in the BBB. However, conventionally developed synthetic nano drug delivery systems still have clear limitations in terms of biocompatibility and safety. In the case of synthetic polymers or inorganic nanoparticles, it has been reported that they are not completely metabolized or excreted in vivo, but instead remain for prolonged periods in major organs such as the liver and spleen, causing chronic cytotoxicity and immune responses. In addition, these artificial delivery vehicles often have low fusion capacity with biological membranes, such that BBB permeation efficiency frequently falls short of expectations, and securing homogeneity during mass production as well as economic feasibility also remain obstacles to commercialization. Accordingly, there is an urgent need to develop a next-generation drug delivery platform that is harmless to the human body, possesses excellent biomembrane permeation performance, and can effectively deliver drugs into the brain without concern for side effects caused by accumulation in the body.

The above description of the background art is provided only for enhancing the understanding the background of the present disclosure and should not be acknowledged as recognizing that it is already well known to those having ordinary knowledge in the art.

### REFERENCES OF RELATED ART

### Non-Patent Documents

(Non-patent document 1) Cohen G, Heikkila E R. The generation of hydrogen peroxide, superoxide radical and hydroxyl radical by 6-hydrosydopamine, dialuric acid, and related cytotoxic agents. J Biol Chem. 1974; 249:2447-52.
(Non-patent document 2) Oxidative Stress: Apoptosis in Neuronal Injury Loh K P, Huang S H, De Silva R, Tan B K, Zhu Y Z. Curr Alzheimer Res. 3(4): 327-337. 2006.
(Non-patent document 3) Information about trend in therapeutic agents and diagnostic technologies for degenerative brain diseases such as dementia, etc., Asan Medical Center, National Institute of Food and Drug Safety Evaluation, 2019.12.

### DISCLOSURE

### Technical Problem

The inventors of the present disclosure have made efforts to find a lactic acid bacteria strain applicable to various kinds of CNS diseases including Alzheimer's disease. As a result, they have identified that a Pediococcus inopinatus strain, a culture thereof or extracellular vesicles derived therefrom have the effect of inhibiting neuroinflammation, which is the cause of various CNS diseases, or protecting neurons, and have completed the present disclosure.

The inventors of the present disclosure have further conducted research to develop a drug delivery technology utilizing strain-derived extracellular vesicles as a carrier capable of crossing the BBB, and have thereby completed the present disclosure.

The present disclosure is directed to providing a food composition for preventing or ameliorating a CNS disease, which contains: Pediococcus inopinatus or a culture, a lysate, an extract or a fermented product thereof; or extracellular vesicles derived from the Pediococcus inopinatus, a culture or the lysate, extract or fermented product thereof; as an active ingredient.

The present disclosure is also directed to providing a pharmaceutical composition for preventing or treating a CNS disease, which contains: Pediococcus inopinatus or a culture, a lysate, an extract or a fermented product thereof; or extracellular vesicles derived from the Pediococcus inopinatus, a culture or the lysate, extract or fermented product thereof; as an active ingredient.

The present disclosure is also directed to providing a method for preventing or treating a CNS disease, which includes a step of administering a therapeutically effective amount of: Pediococcus inopinatus or a culture, a lysate, an extract or a fermented product thereof; or extracellular vesicles derived from the Pediococcus inopinatus, a culture or the lysate, extract or fermented product thereof; to a subject.

The present disclosure is also directed to providing a therapeutic use of a composition containing: Pediococcus inopinatus or a culture, a lysate, an extract or a fermented product thereof; or extracellular vesicles derived from the Pediococcus inopinatus, a culture or the lysate, extract or fermented product thereof

The present disclosure is also directed to providing a method for preparing a composition for preventing, ameliorating or treating a CNS disease, which includes a step of preparing a Pediococcus inopinatus strain.

The present disclosure is also directed to providing a method for preparing a composition for preventing, ameliorating or treating a CNS disease, which includes a step of preparing extracellular vesicles derived from Pediococcus inopinatus or a culture, a lysate, an extract or a fermented product thereof.

The present disclosure is also directed to providing a method for alleviating or treating a CNS disease in a subject in need thereof, comprising administering a therapeutically effective amount of a composition comprising i) extracellular vesicles derived from Pediococcus inopinatus, a culture, a lysate, an extract or a fermented product thereof, and ii) a drug, to a subject, wherein the extracellular vesicles cross the blood-brain barrier (BBB) and deliver the drug to the brain of the subject.

The present disclosure is also directed to providing a method for delivering a drug across the blood-brain barrier (BBB) into the brain of a subject, comprising administering to the subject a composition comprising i) extracellular vesicles derived from Pediococcus inopinatus, a culture, a lysate, an extract or a fermented product thereof, and ii) a drug.

The present disclosure is also directed to providing a composition for crossing the blood-brain barrier (BBB), comprising:
extracellular vesicles derived from Pediococcus inopinatus, a culture, a lysate, an extract or a fermented product thereof, wherein the extracellular vesicles are capable of crossing the BBB.

The present disclosure is also directed to providing a composition for alleviating or treating a CNS disease, comprising:
extracellular vesicles derived from Pediococcus inopinatus, a culture, a lysate, an extract or a fermented product thereof; and
a drug loaded onto or within the extracellular vesicles,
wherein the extracellular vesicles are capable of crossing the blood-brain barrier (BBB) to deliver the drug to the brain.

The present disclosure is also directed to providing a method for preparing a composition for alleviating or treating a CNS disease, comprising:
(a) a step of culturing Pediococcus inopinatus in a culture medium; and
(b) a step of obtaining extracellular vesicles loaded with a drug from the culture of step (a),
wherein the extracellular vesicles loaded with the drug are capable of crossing the blood-brain barrier (BBB) to deliver the drug to the brain.

Other purposes and advantages of the present disclosure will become more apparent by the following detailed description, claims and drawings.

### Technical Solution

In an aspect, the present disclosure provides a composition for preventing, ameliorating or treating a CNS disease, which contains Pediococcus inopinatus or a culture, a lysate, an extract or a fermented product thereof; or extracellular vesicles derived from the Pediococcus inopinatus, a culture or the lysate, extract or fermented product thereof; as an active ingredient.

In another aspect, the present disclosure provides a method for preventing or treating a CNS disease, which includes a step of administering a therapeutically effective amount of: Pediococcus inopinatus or a culture, a lysate, an extract or a fermented product thereof; or extracellular vesicles derived from the Pediococcus inopinatus, a culture or the lysate, extract or fermented product thereof; to a subject.

In another aspect, the present disclosure provides a therapeutic use of a composition containing: Pediococcus inopinatus or a culture, a lysate, an extract or a fermented product thereof; or extracellular vesicles derived from the Pediococcus inopinatus, a culture or the lysate, extract or fermented product thereof.

In another aspect, the present disclosure provides a method for alleviating or treating a CNS disease in a subject in need thereof, comprising administering a therapeutically effective amount of a composition comprising i) extracellular vesicles derived from Pediococcus inopinatus, a culture, a lysate, an extract or a fermented product thereof, and ii) a drug, to a subject, wherein the extracellular vesicles cross the blood-brain barrier (BBB) and deliver the drug to the brain of the subject.

In another aspect, the present disclosure provides a method for delivering a drug across the blood-brain barrier (BBB) into the brain of a subject, comprising administering to the subject a composition comprising i) extracellular vesicles derived from Pediococcus inopinatus, a culture, a lysate, an extract or a fermented product thereof, and ii) a drug.

In another aspect, the present disclosure provides a composition for crossing the blood-brain barrier (BBB), comprising extracellular vesicles derived from Pediococcus inopinatus, a culture, a lysate, an extract or a fermented product thereof, wherein the extracellular vesicles are capable of crossing the BBB.

In another aspect, the present disclosure provides a composition for alleviating or treating a brain disease, comprising:
extracellular vesicles derived from Pediococcus inopinatus, a culture, a lysate, an extract or a fermented product thereof; and
a drug loaded onto or within the extracellular vesicles,
wherein the extracellular vesicles are capable of crossing the blood-brain barrier (BBB) to deliver the drug to the brain.

The inventors of the present disclosure have made efforts to find a lactic acid bacteria strain applicable to various kinds of CNS diseases including Alzheimer's disease. As a result, they have identified that a Pediococcus inopinatus strain, a culture thereof or extracellular vesicles derived therefrom can inhibit neuroinflammation, which is the cause of various CNS diseases, reduce cell death caused by oxidative stress and Aβ (amyloid beta) accumulation or inhibit the activity of acetylcholinesterase, which is the cause of memory impairment.

The drug delivery composition of the present disclosure is attributable to the simple and efficient loading of an antisense oligonucleotide, an siRNA and the like onto or into extracellular vesicles derived from a Pediococcus inopinatus strain, and the excellent capability of the extracellular vesicles to deliver a drug into the brain owing to their permeability across the blood-brain barrier (BBB). Accordingly, the composition can deliver a drug, which was conventionally difficult to deliver into the brain, throughout the brain, including deep regions such as the substantia nigra and the striatum, so that the drug can act functionally therein. Therefore, the drug delivery composition of the present disclosure, as a functional drug delivery platform, is suitable for delivering a variety of CNS therapeutic agents or diagnostic agents, and thus can be utilized as a versatile platform technology extendable to various CNS diseases.

In the present specification, Pediococcus inopinatus specifically refers to a Pediococcus inopinatus strain derived from kimchi.

The Pediococcus inopinatus may be LBMB320040, LBMB320057, LBMB320058, LBMB320061, LBMB320063, LBMB320067, LBMB320069, LBMB320085, LBMB320087, LBMB320090, LBMB320093, LBMB320433, LBMB320503, LBMB320847, LBMB320891, LBMB320908, LBMB320914, LBMB321293, LBMB321610, LBMB321624, LBMB321628, LBMB321673, LBMB321838, LBMB321932, LBMB322483, LBMB322561, LBMB323080, LBMB323081, LBMB323098, LBMB323113, LBMB323133, LBMB323154, or WIKIM27.

Although the Pediococcus inopinatus WIKIM27 strain deposited in the Korean Culture Center of Microorganisms with the accession number KCCM12653P was used as the Pediococcus inopinatus strain in the present disclosure, the means of acquisition is not limited thereto.

In the present specification, the Pediococcus inopinatus strain is a gram-positive facultative anaerobe that can grow under both aerobic and anaerobic conditions. It does not form spores, lacks motility and is sphere-shaped.

In the present specification, the Pediococcus inopinatus strain is a probiotic having the probiotic effect and immune-enhancing effect of general lactic acid bacteria. It is well known that the lactic acid bacteria in the genus Pediococcus inopinatus have probiotic effect and immune-enhancing effect.

In the present specification, the ‘probiotics’ are understood as live microorganisms that provide health benefits by improving the gut microbiota in animals including human. The probiotics are living microorganisms having probiotic activity and can provide beneficial effects to the gut microbiota of the host when provided to human or animals in the form of dried cells or fermented products of single or multiple strains.

In an exemplary embodiment, the present disclosure provides a composition containing Pediococcus inopinatus or a culture, a lysate, an extract or a fermented product thereof.

The Pediococcus inopinatus contained in the composition according to the present disclosure may exist as live or dead bacteria and may also exist in a dried or freeze-dried form. Lactic acid bacteria suitable for various compositions and method for preparing the same are well known to those skilled in the art. For example, the Pediococcus inopinatus may be a culture obtained by culturing in a known liquid or solid medium, a fermented product obtained by culturing the strain with additional ingredients, an extract obtained by extracting the strain with an organic solvent, a lysate obtained by lysing, crushing or homogenizing the cell membrane of the strain, etc., although not being limited thereto.

In a specific exemplary embodiment, the composition may be a composition containing the Pediococcus inopinatus strain existing as live or dead bacteria.

In another specific exemplary embodiment, the composition may be a composition containing a culture, lysate, extract or fermented product of the Pediococcus inopinatus strain.

In another specific exemplary embodiment, the composition may be a composition containing extracellular vesicles derived from Pediococcus inopinatus or a culture, a lysate, an extract or a fermented product thereof.

In another specific exemplary embodiment, the composition is administered via an administration route selected from the group consisting of intravenous administration, intraperitoneal administration, intramuscular administration, subcutaneous administration, intradermal administration, and intranasal administration.

According to an example of the present disclosure, the administering is performed intravenously, thereby delivering the drug to the brain of the subject.

Extracellular vesicles (EVs) allow exchange of substances (proteins, lipids and genetic materials) between cells and function as mediators of physiological/pathological signaling. The extracellular vesicles are largely classified into exosomes and microvesicles. Exosomes have various sizes depending on their origin. They are intraluminal vesicles formed by inward budding of the endosomal membrane during maturation of multi-vesicular endosomes. They are secreted upon fusion of the multi-vesicular endosomes with the cell's plasma membrane. Microvesicles are extracellular vesicles with a size of 50-1,000 nm that are released from the plasma membrane. Individual cells produce different extracellular vesicles depending on physiological state, and secrete extracellular vesicles having specific lipid/protein/nucleic acid compositions (Jae-Wook Lee (2019). Shedding light on the cell biology of extracellular vesicles. BRIC View 2019-R03).

In the present specification, the term “extracellular vesicles” comprise one or more selected from the group consisting of exosomes, microvesicles, ectosomes, and apoptotic bodies.

The exosomes or extracellular vesicles have various diameters in a range of about 1-1,000 nm, specifically 10-1,000 nm, more specifically 10-800 nm, most specifically 20-600 nm.

According to an example of the present disclosure, the exosomes or extracellular vesicles derived from Pediococcus inopinatus according to the present disclosure had a diameter of about 20-600 nm (FIG. 1).

The exosomes or extracellular vesicles contained in the composition of the present disclosure are contained in large quantities in a culture (e.g., a supernatant of the culture) of Pediococcus inopinatus.

The exosomes or extracellular vesicles are contained in a culture (e.g., a supernatant of the culture) of Pediococcus inopinatus at concentrations of 1×10⁴-1×10¹³/mL, specifically 1×10⁵-1×10¹⁰/mL. The exosomes or extracellular vesicles isolated and purified therefrom may be utilized as a therapeutic agent on their own, or a culture, lysate, extract or fermented product containing a large quantity of the exosomes or extracellular vesicles may be utilized as a therapeutic agent.

In another specific exemplary embodiment, the composition may comprise 1×10⁴ to 1×10¹³ of the extracellular vesicles per administration.

In another specific exemplary embodiment, the extracellular vesicles are capable of delivering the drug across the BBB into the brain.

In another specific exemplary embodiment, the extracellular vesicles are retained in the brain of the subject for a period sufficient to deliver the drug to the brain.

The extracellular vesicles may be retained in the brain of the subject for 1 to 14 days, preferably for 1 to 7 days, more preferably for 1 to 12 hours following administration.

According to an exemplary embodiment of the present disclosure, the extracellular vesicles are retained in an organ of the body, particularly the brain, for 1 hour to 12 hours, specifically 2 hours to 6 hours, following intravenous administration, so that a drug loaded therein can be effectively delivered to a target organ during the period of retention (FIG. 13).

According to an exemplary embodiment of the present disclosure, the composition may be excreted via urine.

In another specific exemplary embodiment, the extracellular vesicles may be substantially excreted from the body of the subject via urine within 24 hours following administration.

According to an exemplary embodiment of the present disclosure, the extracellular vesicles are substantially or entirely excreted from the body via urine within 24 hours following intravenous administration, and thus can be effectively utilized for delivery of various drugs without a toxicity concern arising from accumulation in the body (Example 16).

In the present disclosure, the term “drug” refers to any therapeutic, prophylactic, or diagnostic agent capable of exerting a biological or pharmacological effect in a subject upon delivery thereto, and includes, without limitation, a nucleic acid, a peptide, a protein, an antibody, a gene editing agent, a vector, a small molecule compound, a radionuclide, an imaging agent, a diagnostic agent, a peptide nucleic acid (PNA), or a combination thereof.

In the present specification, the term “nucleic acid” refers to a polymer composed of nucleotide monomers and encompasses deoxyribonucleic acid (DNA), ribonucleic acid (RNA), and analogs or modifications thereof. In one embodiment, the nucleic acid is selected from the group consisting of:
(i) an antisense oligonucleotide (ASO), which is a single-stranded oligonucleotide, typically 13 to 25 nucleotides in length, designed to hybridize to a complementary mRNA sequence and modulate gene expression by RNase H-mediated degradation of the target mRNA, steric blocking of translation, or splicing modulation. ASOs include, but are not limited to, gapmers, mixmers, and steric-blocking oligonucleotides, and may comprise chemical modifications such as phosphorothioate backbone modifications, 2′-O-methyl, 2′-O-methoxyethyl(2′-MOE), or locked nucleic acid (LNA) modifications;
(ii) a small interfering RNA (siRNA), which is a double-stranded RNA molecule, typically 19 to 23 base pairs in length, that induces sequence-specific post-transcriptional gene silencing through the RNA interference (RNAi) pathway via the RNA-induced silencing complex (RISC);
(iii) a short hairpin RNA (shRNA), which is a single-stranded RNA molecule that forms a tight hairpin structure and is processed intracellularly into siRNA to silence target gene expression via the RNAi pathway;
(iv) a double-stranded RNA (dsRNA), which is a double-stranded ribonucleic acid molecule capable of triggering RNA interference or innate immune responses;
(v) a messenger RNA (mRNA), which is a single-stranded RNA molecule encoding a protein of interest, capable of being translated by cellular ribosomes to produce a therapeutic protein upon delivery to target cells;
(vi) a microRNA (miRNA), which is a short non-coding RNA molecule, typically 18 to 25 nucleotides in length, that regulates gene expression at the post-transcriptional level by binding to the 3′-untranslated region (3′-UTR) of target mRNAs;
(vii) a single guide RNA (sgRNA) or CRISPR component, which includes guide RNAs used in conjunction with CRISPR-associated proteins (e.g., Cas9, Cas12, or Cas13) for sequence-specific genome editing, gene activation, or gene repression;
(viii) a DNA, which includes single-stranded or double-stranded deoxyribonucleic acid molecules, including plasmid DNA, linear DNA, or oligodeoxynucleotides, used for gene expression, gene correction, or diagnostic purposes; and
(ix) an aptamer, which is a short single-stranded oligonucleotide (DNA or RNA) that folds into a specific three-dimensional structure capable of binding to a target molecule, such as a protein, small molecule, or cell, with high affinity and specificity.

In the present specification, the term “peptide” refers to a short chain of amino acid residues linked by peptide bonds, typically comprising 2 to 50 amino acids, including cell-penetrating peptides, neuropeptides, and therapeutic peptides.

In the present specification, the term “protein” refers to a polypeptide chain comprising more than 50 amino acid residues, including enzymes, growth factors, cytokines, and other functional proteins having therapeutic activity.

In the present specification, the term “antibody” refers to an immunoglobulin molecule or a fragment or derivative thereof that specifically binds to a target antigen. The antibody includes, but is not limited to, a full-length antibody, a monoclonal antibody, a polyclonal antibody, a humanized antibody, a chimeric antibody, a bispecific antibody, and antigen-binding fragments thereof such as Fab, Fab′, F(ab′)₂, Fv, and single-chain variable fragment (scFv).

In the present specification, the term “gene editing agent” refers to any agent capable of modifying the genomic sequence of a target cell, including but not limited to CRISPR-Cas9 ribonucleoprotein complexes (RNPs), zinc finger nucleases (ZFNs), transcription activator-like effector nucleases (TALENs), and base editors.

In the present specification, the term “small molecule compound” refers to a low molecular weight (typically less than 900 daltons) organic or inorganic compound that can modulate a biological process. Small molecule compounds include, but are not limited to, chemotherapeutic agents, kinase inhibitors, neurotransmitter modulators, and anti-inflammatory agents.

In the present specification, the term “radionuclide” refers to a radioactive isotope that emits ionizing radiation and can be used for therapeutic purposes (e.g., targeted radionuclide therapy) or diagnostic imaging (e.g., PET or SPECT imaging). Examples include ¹³¹I, ⁹⁰Y, ¹⁷⁷Lu, ⁶⁴Cu, and ⁶⁸Ga.

In the present specification, the term “imaging agent” refers to a substance used to enhance contrast or signal in diagnostic imaging, including but not limited to fluorescent dyes, near-infrared (NIR) fluorescent probes, magnetic resonance imaging (MRI) contrast agents, and positron emission tomography (PET) tracers. The use of imaging agents loaded within the extracellular vesicles according to the present disclosure enables non-invasive visualization of drug distribution in the brain.

In the present specification, the term “diagnostic agent” refers to a substance used for detecting, identifying, or monitoring a disease or biological condition, and includes, but is not limited to, biomarker-targeting agents, molecular probes, and contrast agents.

In the present specification, the term “peptide nucleic acid (PNA)” refers to a synthetic nucleic acid analog in which the naturally occurring phosphodiester backbone is replaced by a polyamide backbone composed of N-(2-aminoethyl)glycine units. PNA hybridizes to complementary DNA or RNA sequences with high affinity and specificity, and is resistant to nuclease and protease degradation, making it suitable for gene silencing and diagnostic applications.

In the present specification, the term “vector” refers to a nucleic acid molecule or particle used as a vehicle to deliver genetic material or a therapeutic agent into a target cell. The vector includes, but is not limited to:
(i) a viral vector, which is derived from a virus and utilizes the natural ability of viruses to enter cells and deliver genetic cargo. Viral vectors include, but are not limited to, adeno-associated virus (AAV) vectors, adenoviral vectors, lentiviral vectors, retroviral vectors, herpes simplex virus (HSV) vectors, and baculoviral vectors. In a specific embodiment, the viral vector is an AAV vector, which is particularly suitable for CNS gene therapy due to its ability to transduce non-dividing neurons, its low immunogenicity, and its capacity for long-term transgene expression. AAV serotypes applicable to brain-targeted delivery include, but are not limited to, AAV1, AAV2, AAV5, AAV8, AAV9, AAVrh10, and AAV-PHP.eB; and
(ii) a non-viral vector, which includes synthetic or naturally derived particles used for nucleic acid delivery without a viral component, including but not limited to plasmid vectors, lipid nanoparticle (LNP)-based vectors, polymeric vectors, and cationic liposome-based vectors.

The drug may be loaded onto or within the extracellular vesicles by any method known in the art. In one embodiment, the drug is loaded by co-culturing the Pediococcus inopinatus strain together with the drug in a culture medium such that the drug is incorporated into the extracellular vesicles during the co-culturing. In another embodiment, the drug is loaded by incubating previously isolated extracellular vesicles with the drug under conditions sufficient for the drug to be incorporated into the extracellular vesicles, followed by removal of unincorporated drug by centrifugation or filtration. The number of drug types loaded into a single extracellular vesicle is not particularly limited, and one type or two or more types of drugs may be simultaneously loaded onto or within the extracellular vesicles.

In another specific exemplary embodiment, the drug may be a RNA interference-inducing nucleic acid molecule.

According to an example of the present disclosure, the drug is an antisense oligonucleotide (ASO) that targets mRNA encoding alpha-synuclein, and/or the administering reduces alpha-synuclein protein accumulation in the substantia nigra of the subject (Examples 18-20).

According to an example of the present disclosure, the composition preserves dopaminergic neurons in the striatum and substantia nigra of the subject (Example 21).

According to an example of the present disclosure, the drug is an siRNA that targets mRNA encoding a neuropeptide.

According to an example of the present disclosure, the neuropeptide may be selected from the group consisting of agouti-related peptide (AgRP) and neuropeptide Y (NPY).

According to an example of the present disclosure, the drug is an siRNA that targets mRNA encoding a neuropeptide selected from the group consisting of agouti-related peptide (AgRP) and neuropeptide Y (NPY) (Example 22).

In the present disclosure, the term “neuropeptide” refers to a small protein or peptide molecule produced and released by neurons, which acts as a neurotransmitter or neuromodulator to regulate neuronal signaling and physiological functions such as appetite, mood, or pain, and includes, without limitation, agouti-related peptide (AgRP) and neuropeptide Y (NPY).

RNA interference (RNAi) is induced by small double-stranded RNA molecules such as siRNA (small interfering RNA) or shRNA (short hairpin RNA), and refers to a mechanism in which a nucleotide sequence complementary to a target mRNA sequence forms an RNA-induced silencing complex (RISC), thereby degrading the target mRNA or inhibiting its translation. siRNA has a relatively short double-stranded structure (typically a double-stranded region of 19 to 23 nt with 3′ overhangs at both ends), and is being developed as a therapeutic agent against therapeutic target genes for various diseases owing to its high target specificity and potent knock-down efficacy (P. J. Kamola et al., “The siRNA non-seed region and its target sequences are auxiliary determinants of off-target effects,” PLoS Comput. Biol., 2015; Myers et al., “Minimizing off-target effects by using diced siRNAs for RNA interference,” Journal of RNAi and Gene Silencing, 2006).

In the present disclosure, the term “RNA interference (RNAi)” refers to a phenomenon in which the expression of a specific gene is reduced as a result of degradation of a target mRNA or inhibition of its translation by a double-stranded RNA or an analogue thereof, and includes mechanisms mediated by siRNA, shRNA, miRNA, dsRNA, or a chemical derivative thereof.

In the present disclosure, the term “RNA interference-inducing nucleic acid molecule” refers to a double-stranded construct comprising an antisense strand complementary to the sequence of an mRNA of a target molecule and a sense strand complementary to at least a portion of the antisense strand, and includes a small interfering RNA (siRNA), a Dicer-substrate siRNA (DsiRNA), a short hairpin RNA (shRNA), an shRNA-derived siRNA, an miRNA analogue, a dsRNA fragment, a double-stranded structure in a PNA form, and a chemically modified version thereof.

The siRNA is typically composed of an antisense strand of about 7 to 30 nucleotides in length and a sense strand complementary thereto. The siRNA may have a structure in which 1 to 5, preferably 1 to 3, nucleotide overhangs are present or absent at the 3′ end of each strand, and may include a double-stranded region within a range of 7 to 30 nucleotides in length, preferably 15 to 27 nucleotides in length, more preferably 19 to 23 nucleotides in length, and even more preferably 19 to 21 nucleotides in length.

In the present disclosure, the term “antisense strand” or “guide strand” refers to a strand comprising a nucleotide sequence complementary to a specific target sequence on the mRNA of a target molecule, which is loaded onto the RNA-induced silencing complex (RISC) to recognize and cleave the target mRNA, and may, if necessary, comprise a phosphate group or a phosphate mimic at the 5′ end.

In the present disclosure, the term “sense strand” or “passenger strand” refers to a strand comprising a nucleotide sequence complementary to the antisense strand so as to form a double-stranded structure, but which does not perform the primary guide function in the RISC, and may include a modified nucleotide or a terminal modification.

According to one exemplary embodiment of the present disclosure, the siRNA comprised in the EV of the present disclosure may have the nucleic acid sequence of SEQ ID NO: 3 or 4.

The antisense oligonucleotide may have a length of about 8 to 50 nucleotides, preferably about 12 to 30 nucleotides, more preferably about 14 to 22 nucleotides, and may comprise one or more chemically modified nucleotides and/or an internucleotide linkage modification to enhance nuclease resistance, binding affinity to the target mRNA, and/or pharmacokinetic properties. Examples of such chemical modifications include, but are not limited to, a phosphorothioate backbone linkage, a 2′-O-methyl(2′-OMe) modification, a 2′-O-methoxyethyl(2′-MOE) modification, a locked nucleic acid (LNA), a constrained ethyl (cEt) modification, a peptide nucleic acid (PNA), and a phosphorodiamidate morpholino oligomer (PMO).

In a specific exemplary embodiment, the antisense oligonucleotide may be a gapmer-type antisense oligonucleotide comprising a central DNA or DNA-like segment capable of recruiting RNase H to cleave the target mRNA, flanked at both the 5′ and 3′ ends by wing segments comprising a chemically modified nucleotide that confers enhanced nuclease resistance and binding affinity.

According to an example of the present disclosure, the antisense oligonucleotide is a RNase H-mediated gapmer ASO that directly targets an mRNA encoding alpha-synuclein.

According to one exemplary embodiment of the present disclosure, the ASO comprised in the EV of the present disclosure may have the nucleic acid sequence of SEQ ID NO: 1 or 2.

According to an example of the present disclosure, the drug is loaded onto or within the extracellular vesicles.

In the present disclosure, the term “isolation” includes not only a process of selectively obtaining a desired substance (e.g., exosomes) from a biological sample (e.g., a Pediococcus inopinatus culture) (positive isolation) but also a process of selectively removing impurities other than the desired substance (negative isolation). Therefore, the term “isolation” may be used with the same meaning as “obtainment”, “extraction” or “purification”. In the present disclosure, the isolation of exosomes or extracellular vesicles may be performed by any method commonly employed in the art without limitation. For example, a commercially available exosome isolation kit (e.g., EXO-BB, ExoQuick®-ULTRA, ExoQuick®-TC, Capturem™ exosome isolation kit, total exosome isolation kit, ExoTrap™ exosome isolation spin column kit, Exo2D™, etc.) may be utilized, or separation based on the difference in the specific gravity of ingredients in a solution (e.g., centrifugation), separation based on size (e.g., ultrafiltration or vacuum filtration) or separation based on the affinity for a specific substrate (e.g., affinity chromatography) may be included. However, any separation method based on the intrinsic physical properties of a desired substance in a nonhomogeneous sample commonly used in the art may be used without limitation.

In another aspect, the present disclosure provides a probiotic composition containing: Pediococcus inopinatus or a culture, a lysate, an extract or a fermented product thereof; or extracellular vesicles derived from the Pediococcus inopinatus, a culture or the lysate, extract or fermented product thereof.

In the present specification, the probiotic composition may be used to prevent, treat or ameliorate a gastrointestinal disease of animals including human. Specifically, the animal includes livestock such as cow, horse and pig. The ‘gastrointestinal disease’ includes both infection by gastroenteric harmful bacteria and inflammatory bowel disease. For example, it includes infectious diarrhea caused by pathogenic microorganisms (E. coli, Salmonella, Clostridium, etc.), gastrointestinal inflammation, inflammatory bowel disease, neurogenic enteritis syndrome, overproliferation of intestinal microorganisms, acute diarrhea, etc., although not being limited thereto.

Specifically, the probiotic composition according to the present disclosure is administered orally. The administration dosage may vary depending on the particular gastrointestinal disease, the severity of the disease, age, sex, race, purpose such as treatment or prevention, etc. In general, 10 million to 100 billion cells may be administered a day for an adult.

In another aspect, the present disclosure provides a composition for enhancing immunity, which contains: Pediococcus inopinatus or a culture, a lysate, an extract or a fermented product thereof; or extracellular vesicles derived from Pediococcus inopinatus or a culture, a lysate, an extract or a fermented product thereof.

According to a specific exemplary embodiment of the present disclosure, the composition of the present disclosure is a food composition.

The Pediococcus inopinatus or a culture, a lysate, an extract or a fermented product thereof; or the extracellular vesicles derived from the Pediococcus inopinatus, a culture or the lysate, extract or fermented product thereof contained in the food composition of the present disclosure is the same as described above.

When the composition of the present disclosure is used as a food composition, the food composition may be in the form of a functional health food, a condiment, a beverage, a bar, etc. In addition, the food composition containing the strain as an active ingredient may be a beverage such as fermented milk, etc. Therefore, the present disclosure provides a lactic acid bacteria starter for fermenting food, which contains Pediococcus inopinatus or a culture, a lysate, an extract or a fermented product thereof; or extracellular vesicles derived from the Pediococcus inopinatus, a culture or the lysate, extract or fermented product thereof.

The food composition of the present disclosure may be prepared using, in addition to the active ingredient, a sitologically suitable and physiologically acceptable adjuvant. The adjuvant may be an excipient, a disintegrant, a sweetener, a binder, a coating agent, a swelling agent, a lubricant, a glidant, a flavorant, etc.

Specifically, the food composition may be prepared using one or more sitologically acceptable carrier in addition to the active ingredient described above.

For example, for preparation of a tablet or a capsule, the active ingredient may be bound with an oral, nontoxic, pharmaceutically acceptable inert carrier such as ethanol, glycerol, water, etc. In addition, if desired or necessary, a suitable binder, lubricant, disintegrant and coloring agent may also be added. Suitable binders include natural sugar such as starch, gelatin, glucose or β-lactose, corn sweetener, natural or synthetic gum such as acacia, tragacanth or sodium oleate, sodium stearate, magnesium stearate, sodium benzoate, sodium acetate, sodium chloride, etc., although not being limited thereto. The disintegrant includes starch, methyl cellulose, agar, bentonite, xanthan gum, etc., although not being limited thereto. When the composition is prepared as a liquid solution, one or more of saline, sterile water, Ringer's solution, buffered saline, albumin injection, dextrose solution, maltodextrin solution, glycerol and ethanol may be used as an acceptable pharmaceutical carrier and, if necessary, other common additives such as an antioxidant, a buffer, a bacteriostat, etc. may be used. In addition, an injectable formulation such as an aqueous solution, a suspension, an emulsion, etc., a pill, a capsule, a granule, or a tablet may be prepared by further adding a diluent, a dispersant, a surfactant, a binder or a lubricant.

The food composition according to the present disclosure may be added to various foods. The foods to which the composition of the present disclosure may be added include, for example, beverages, vitamin complexes, health supplements, etc.

The food composition of the present disclosure may contain the ingredients commonly used for preparation of food, e.g., a protein, a carbohydrate, a fat, a nutrient, a seasoning agent and a flavorant. Examples of the carbohydrate include common sugars such as monosaccharides, e.g., glucose, fructose, etc., disaccharides, e.g., maltose, sucrose, oligosaccharide, etc. and polysaccharides, e.g., dextrin, cyclodextrin, etc. and sugar alcohols such as xylitol, sorbitol, erythritol, etc. Examples of the flavorant include natural flavorants [thaumatin, stevia extract (e.g., rebaudioside A, glycyrrhizin, etc.)] and synthetic flavorants (saccharin, aspartame, etc.). For example, when the food composition of the present disclosure is prepared as a drink or a beverage, it may further contain citric acid, fructose syrup, sugar, glucose, acetic acid, malic acid, fruit juice, plant extracts, etc.

According to a specific exemplary embodiment of the present disclosure, the composition is a pharmaceutical composition.

In addition, the present disclosure provides a method for preventing or treating a CNS disease, which includes a step of administering a therapeutically effective amount of: Pediococcus inopinatus or a culture, a lysate, an extract or a fermented product thereof; or extracellular vesicles derived from the Pediococcus inopinatus, a culture or the lysate, extract or fermented product thereof; to a subject.

In addition, the present disclosure provides a use of a composition containing: Pediococcus inopinatus or a culture, a lysate, an extract or a fermented product thereof; or extracellular vesicles derived from the Pediococcus inopinatus, a culture or the lysate, extract or fermented product thereof; for treating a CNS disease.

In the present specification, the “subject” refers to a mammal which is a subject of treatment, monitoring or experiment. Specifically, it may be human or an animal in need of prevention and/or treatment of a CNS disease.

In the present disclosure, the term “CNS disease” refers to any disease, disorder, or condition involving structural or functional abnormality of the brain or central nervous system, including, without limitation, a cerebrovascular disease (e.g., stroke, cerebral infarction, cerebral hemorrhage, or cerebral aneurysm), a degenerative brain disease (e.g., Alzheimer's disease, vascular dementia, Parkinson's disease, or amyotrophic lateral sclerosis), neurogenetic diseases (e.g., Dravet syndrome, SMA (Spinal Muscular Atrophy), Angelman syndrome, Rett syndrome, Spinocerebellar ataxia), an inflammatory brain disease (e.g., Multiple sclerosis, Neuromyelitis optica spectrum disorder (NMOSD)), epilepsy, a CNS tumor (e.g., glioblastoma, Astrocytoma, Medulloblastoma, Brain metastases, Spinal cord tumors), and headache or dizziness. The CNS disease may be caused by neuronal death, neuroinflammation, abnormal protein accumulation, impaired neurotransmission, or a combination thereof.

According to a specific exemplary embodiment of the present disclosure, the CNS disease is a degenerative brain disease.

The “degenerative brain disease” may be one or more disease selected from a group consisting of Parkinson's disease, Alzheimer's disease, Huntington's disease, Lou Gehrig's disease (amyotrophic lateral sclerosis), Creutzfeldt-Jakob disease, stroke, multiple sclerosis, learning disorder, cognitive impairment, neuroinflammation, neuronal damage and memory impairment, although not being limited thereto.

According to a specific exemplary embodiment of the present disclosure, the degenerative brain disease is Alzheimer's disease.

According to a specific exemplary embodiment of the present disclosure, the degenerative brain disease is neuroinflammation.

According to a specific exemplary embodiment of the present disclosure, the degenerative brain disease is neuronal damage.

According to a specific exemplary embodiment of the present disclosure, the degenerative brain disease is memory impairment.

According to a specific exemplary embodiment of the present disclosure, the degenerative brain disease is learning disorder.

According to a specific exemplary embodiment of the present disclosure, the degenerative brain disease is cognitive impairment.

According to a specific exemplary embodiment of the present disclosure, the composition inhibits the activity of acetylcholinesterase (AChE).

The correlation between neuroinflammation and the onset of degenerative brain diseases has been known through a number of literatures (Mol Med Rep. 2016 Apr. 13 (4): 3391-3396; J Immunol Res. 2018 Apr. 16, 2018:4784268; Trans/Neurodegener. 2015, 4:19, etc.), and the technology of using neuroinflammatory regulators for treating degenerative brain disease is also known wells.

In addition, oxidative stress-induced cell death has been reported as the cause of various degenerative brain diseases including Alzheimer's disease and stroke (Kok Poh Loh et al., Oxidative Stress: Apoptosis in Neuronal Injury, Curr Alzheimer Res. 3 (4): 327-337. 2006).

The composition of the present disclosure may be utilized as an agent for preventing, ameliorating or treating various degenerative brain diseases including Alzheimer's disease and stroke since it inhibits neuroinflammation, which is the cause of the degenerative brain diseases, or reduces cell death induced by oxidative stress and Aβ (amyloid beta) accumulation.

In the present specification, the “memory impairment” or “cognitive impairment” refers to memory decline, memory disorder or cognitive decline occurring as the brain shrinks and brain cells are destroyed due to physical fatigue, insufficient sleep, excessive alcohol intake, dementia and other CNS diseases. The prevention, amelioration or treatment of “memory impairment” or “cognitive impairment” refers to the effect of maintaining cognitive ability by regulating harmful substances that damage brain cells or improving declined cognitive ability by regulating neurotransmitters of the brain. The memory refers to the ability of storing information in the brain and retrieving it when necessary, and the cognitive ability refers to the ability of differentiating and recognizing things.

The pharmaceutical composition according to the present disclosure may be administered orally or parenterally.

The parenteral administration can be achieved, for example, by intravenous injection, transdermal administration, subcutaneous injection, intramuscular injection, intravitreal injection, eye drop administration, intracerebroventricular injection, intrathecal injection, intraamniotic injection, intraarterial injection, intraarticular injection, intracardiac injection, intracavernous injection, intracerebral injection, intracisternal injection, intracoronary injection, intracranial injection, intradural injection, epidural injection, intrahippocampal injection, intranasal injection, intraosseous injection, intraperitoneal injection, intrapleural injection, intraspinal injection, intrathoracic injection, intrathymic injection, intrauterine injection, intravaginal injection, intraventricular injection, intravesical injection, subconjunctival injection, intratumoral injection, topical injection, etc.

The pharmaceutical composition of the present disclosure may contain a pharmaceutically acceptable carrier. In the present disclosure, the term “pharmaceutically acceptable carrier” refers to a carrier or diluent which does not cause significant irritation to an organism and does not abrogate the biological activity and properties of the administered ingredient. In the present disclosure, the pharmaceutically acceptable carrier may be one or more of saline, sterile water, Ringer's solution, buffered saline, dextrose solution, maltodextrin solution, glycerol and ethanol. If necessary, an injectable formulation suitable for injection to tissues or organs may be prepared by adding commonly used other additives such as an antioxidant, a buffer, a bacteriostat, etc. In addition, it can be formulated into a dried preparation (particularly, a freeze-dried preparation) that can be prepared into an injectable solution by adding an isotonic sterile solution or, in some cases, sterile water or physiological saline. In addition, a target organ-specific antibody or ligand can be bound to the carrier so as to allow specific action on the target organ. Suitable preparations well known in the art are disclosed in Remington's Pharmaceutical Science (Mack Publishing Company, Easton PA).

Specifically, the composition of the present disclosure may further contain a filler, an excipient, a disintegrant, a binder, a glidant, etc. In addition, the composition of the present disclosure may be formulated by a method well known in the art so as to provide quick, sustained or delayed release of the active ingredient after administration to a mammal.

In the present disclosure, the “administration” refers introduction of the composition of the present disclosure to a patient by any suitable means. The composition of the present disclosure may be administered through various oral or parenteral routes as long as it can reach the target tissue.

For example, the composition of the present disclosure may be administered clinically by intramuscular, intravenous, intranasal or intraperitoneal injection.

For injection, it may be specifically formulated into a pharmaceutically suitable buffer such as Hank solution, Ringer solution or physiological saline buffer. For transmucosal administration, a penetrant suitable for the corresponding barrier is used. Such penetrants are generally known in the art.

Formulations for parenteral administration include a sterilized aqueous solution, a nonaqueous solution, a suspension, an emulsion, etc. For the nonaqueous solution or suspension, propylene glycol, polyethylene glycol, vegetable oil such as olive oil, an injectable ester such as ethyl oleate, etc. may be used.

In the present specification, the “effective amount” or “therapeutically effective amount” refers to an amount necessary to delay or completely stop the onset or progress of a specific disease desired to be treated, and the effective amount of Pediococcus inopinatus contained in the food composition or pharmaceutical composition of the present disclosure means the amount required to achieve the effect of preventing, ameliorating or treating a CNS disease. Accordingly, the effective amount may be controlled depending on various factors including the kind of the disease, the severity of the disease, the kinds and contents of other ingredients contained in the composition, the age, body weight, general health condition and diet of a patient, administration time, administration route, treatment period, and co-administered drugs. It is obvious to those skilled in the art that an appropriate daily administration dosage can be determined adequately by a physician.

For the purpose of the present disclosure, it is desired that the therapeutically effective amount for a particular patient is varied depending on various factors such as the response desired to be achieved, the particular composition, the age, body weight, general health condition, sex and diet of a patient, administration time, administration route, the excretion rate of the composition, treatment period, and co-administered drugs, and other similar factors well known in the medical field.

In the present specification, the “treatment” refers to an approach to achieve a favorable or desired clinical result. For the purpose of the present disclosure, the favorable or desired clinical result includes nonrestrictively the amelioration of symptoms, the reduction in the reduction of the scope of diseases, the stabilization (i.e., prevention of aggravation) of disease state, the delay or slowing of disease progress, and the amelioration or temporary alleviation and reduction of disease state (partially or completely). In addition, the “treatment” may also mean increasing survival rate as compared to that expected when the treatment is not given. The “alleviation” of a disease refers to the reduction of the scope of disease state and/or undesired clinical symptoms and/or the delay or extension of the time course of progress as compared to the absence of the treatment.

The pharmaceutical composition according to an exemplary embodiment of the present disclosure may be administered together with one or more substances exhibiting activity against a CNS disease.

In addition, the pharmaceutical composition according to an exemplary embodiment of the present disclosure may be used alone, or in combination with a method using surgery, hormone therapy, drug therapy and/or a biological response modifier, for preventing or treating a CNS disease.

In another aspect, the present disclosure provides a feed additive or a feed containing: Pediococcus inopinatus or a culture, a lysate, an extract or a fermented product thereof; or extracellular vesicles derived from the Pediococcus inopinatus, a culture or the lysate, extract or fermented product thereof; as an active ingredient.

When used as a feed additive, the composition may be prepared in the form of a 20-90% concentrate, powder or granule. The feed additive may further contain one or more of an organic acid such as citric acid, fumaric acid, adipic acid, lactic acid, malic acid, etc., a phosphate such as sodium phosphate, potassium phosphate, acid pyrophosphate, polyphosphate, etc., and a natural antioxidant such as polyphenol, catechin, α-tocopherol, rosemary extract, vitamin C, green tea extract, licorice extract, chitosan, tannic acid, phytic acid, etc. When used as a feed, the composition may be formulated into a common feed form and may contain common feed ingredients.

The feed additive and feed may further contain a dried ingredient selected from a grain, e.g., ground or crushed wheat, oats, barley, corn or rice, a vegetable protein feed, e.g., a feed having rape, bean and sunflower as main ingredients, an animal protein feed, e.g., blood meal, meat meal, bone meal or fish meal, a sugar, a dairy product, e.g., powdered milk or whey powder, etc., and may further contain a nutritional supplement, a digestion- and absorption-improving agent, a growth-promoting agent, etc.

The feed additive may be administered to an animal either alone or in combination with another feed additive in an edible carrier. In addition, the feed additive may be easily administered to an animal by directly mixing with an animal feed as a top dressing or as an oral formulation separately from the feed. When the feed additive is administered separately from the animal feed, it may be prepared as an immediate or sustained release formulation in combination with a sitologically acceptable edible carrier, as well known in the art. The edible carrier may be solid or liquid, for example, cornstarch, lactose, sucrose, bean flake, peanut oil, olive oil, sesame oil and propylene glycol. When a solid carrier is used, the feed additive may be a tablet, a capsule, a powder, a troche, a sugar-coated tablet or a non-dispersed top dressing. When used as a liquid carrier, the feed additive may be a soft gelatin capsule, a syrup, a suspension, an emulsion or a solution.

In addition, the feed additive and the feed may contain an adjuvant, e.g., a preservative, a stabilizer, a wetting agent, an emulsifier, a solubilizer, etc. The feed additive may be used by adding to an animal feed by spraying or mixing.

The feed or feed additive of the present disclosure may be applied to animal feeds for mammals, poultry and fish.

It may be used for mammals such as pig, cow, sheep, goat, experimental rodents, pets (e.g., dog or cat), etc., poultry such as chicken, turkey, duck, goose, pheasant, quail, etc., and fish such as trout, etc., although not being limited thereto.

The feed or feed additive of the present disclosure may be used in animal feeds for enhancing the growth, immunity, etc. of animals.

The amount of the Pediococcus inopinatus strain contained in the composition according to the present disclosure may be about 10⁶-10¹² cfu/mL, e.g., 10⁷-10¹¹ cfu/mL or 10⁸-10¹⁰ cfu/mL, for a single administration. Specifically, the strain may be administered in the form of live bacteria, and may be killed or attenuated before administration. In addition, when the composition is prepared using a supernatant of a culture, it may pass through an additional sterilization process by heating. The amount of the strain necessary to provide minimum effect may vary depending on the physical or health condition of a subject. In general, a daily administration dosage may be about 10⁶-10¹² cfu/mL, e.g., 10⁷-10¹¹ cfu/mL or 10⁸-10¹⁰ cfu/mL.

In another aspect, the present disclosure provides a method for preparing a composition for preventing, ameliorating or treating a CNS disease, which includes:
(a) a step of preparing a Pediococcus inopinatus strain; and
(b) a step of culturing the strain in a culture medium.

According to a specific exemplary embodiment of the present disclosure, the preparation method further includes a step of separating extracellular vesicles from the strain or the culture medium.

In another aspect, the present disclosure provides a method for preparing a composition for preventing, ameliorating or treating a CNS disease, which includes a step of preparing extracellular vesicles derived from the Pediococcus inopinatus strain.

In another aspect, the present disclosure provides a method for preparing a composition for alleviating or treating a CNS disease, comprising:
(a) a step of culturing Pediococcus inopinatus in a culture medium; and
(b) a step of obtaining extracellular vesicles loaded with a drug,
wherein the extracellular vesicles loaded with the drug are capable of crossing the blood-brain barrier (BBB) to deliver the drug to the brain.

In another specific exemplary embodiment, the step (b) may comprise isolating extracellular vesicles from the culture of step (a).

In another specific exemplary embodiment, the step (b) may comprise loading a drug into or onto the isolated extracellular vesicles.

In another specific exemplary embodiment, the step (b) may comprise:
co-culturing the Pediococcus inopinatus together with the drug such that the drug is incorporated into the extracellular vesicles during the co-culturing; and
isolating the extracellular vesicles loaded with the drug from the culture.

In another specific exemplary embodiment, the step (b) may comprise:
isolating extracellular vesicles from the culture of step (a); and
incubating the isolated extracellular vesicles with the drug under conditions sufficient for the drug to be incorporated into the extracellular vesicles, thereby obtaining the extracellular vesicles loaded with the drug.

In the present disclosure, the term “loading” refers to a process by which a drug becomes associated with an extracellular vesicle, whether by encapsulation within the lumen of the extracellular vesicle, insertion into or attachment to the membrane of the extracellular vesicle, expression of a therapeutic protein on the membrane of the extracellular vesicle, or expression of a therapeutic protein fused to, or otherwise bound to, a membrane protein of the extracellular vesicle (e.g., an exosomal marker protein), or a combination thereof, such that the drug is co-isolated and co-delivered together with the extracellular vesicle. The drug may be loaded onto or into the extracellular vesicle during biogenesis of the extracellular vesicle in a producing cell, or after isolation of the extracellular vesicle, without limitation. For example, where the drug is a protein or peptide, the therapeutic agent may be associated with the extracellular vesicle through physical absorption, chemical conjugation, membrane association or other suitable loading methods.

In the present disclosure, the term “co-culturing” refers to a process of culturing the Pediococcus inopinatus strain in the presence of a drug under conditions suitable for growth of the strain and production of extracellular vesicles, such that the drug present in the culture medium becomes loaded onto or into the extracellular vesicles during their biogenesis and secretion.

The advantages and features of the present disclosure and the methods for achieving the same will become more apparent by the examples described below. However, the present disclosure is not limited by the examples but may be embodied in various different forms. The examples are provided so that the disclosure of the present disclosure is complete and the scope of the present disclosure can be clearly understood by those having ordinary knowledge in the art to which the present disclosure belongs, and the scope of the present disclosure is defined only by the appended claims.

### Advantageous Effects

The features and advantages of the present disclosure may be summarized as follows:
(i) The present disclosure provides a composition for preventing, ameliorating or treating a CNS disease, which contains: Pediococcus inopinatus or a culture, a lysate, an extract or a fermented product thereof; or extracellular vesicles derived from the Pediococcus inopinatus, a culture or the lysate, extract or fermented product thereof; as an active ingredient.
(ii) The composition of the present disclosure inhibits neuroinflammation, which is a cause of a degenerative brain disease, or reduces cell death caused by oxidative stress and Aβ accumulation. Therefore, it can be utilized as an agent for preventing, ameliorating or treating various degenerative brain diseases including Alzheimer's disease and stroke.
(iii) The extracellular vesicles derived from the Pediococcus inopinatus strain loaded with a drug according to the present disclosure are capable of crossing the blood-brain barrier (BBB), and thus can deliver the drug to the whole brain, including deep regions thereof, in vivo by mere intravenous administration. In addition, since the drug can be efficiently loaded onto the extracellular vesicles even under simple strain culture conditions, the extracellular vesicles may provide an alternative to conventional invasive direct CNS administration methods, including intracerebral injection, intracerebroventricular injection (ICV) and stereotaxic injection, and can thus be utilized as a platform technology effectively applicable to improving patient compliance and establishing long-term treatment strategies for CNS diseases and the like.

### BRIEF DESCRIPTION OF DRAWINGS

FIG. 1 shows an SEM (scanning electron microscopy) image showing the surface structure of a Pediococcus inopinatus LB-400 strain from the supernatant of a culture obtained by culturing the strain for 24 hours.

FIG. 2 schematically illustrates an experimental procedure of identifying the anti-inflammatory effect of a culture of a Pediococcus inopinatus LB-400 strain in BV-2 cells, which are microglial cells involved in neuroinflammation in the brain.

FIG. 3 schematically illustrates an experimental procedure of identifying the neuron-protecting effect of a Pediococcus inopinatus LB-400 strain and a culture thereof in SH-SY5Y cells, which are neuroblastoma cells involved in neuronal death in the brain.

FIGS. 4A and 4B show a result of measuring the inflammatory marker NO (nitric oxide) released when microglia in which inflammation is induced by LPS are treated with a culture of a Pediococcus inopinatus LB-400 strain of the present disclosure (FIG. 4A) or extracellular vesicles (EVs) derived from the LB-400 strain (FIG. 4B) by Griess assay at 540 nm.

FIG. 5 shows a result of investigating the cytotoxicity of a culture of a Pediococcus inopinatus LB-400 strain of the present disclosure in microglia in which inflammation is induced by LPS by WST-8 assay at 450 nm.

FIG. 6 shows a result of investigating the protection of nerves from oxidative stress in neuroblastoma in which oxidative stress-induced cell death is induced by H₂O₂ (hydrogen peroxide) by a Pediococcus inopinatus LB-400 strain of the present disclosure by WST assay at 450 nm.

FIGS. 7A-7C show a result of investigating the protection of nerves from oxidative stress in neuroblastoma in which oxidative stress-induced cell death is induced by 6-OHDA (6-hydroxydopamine) by a Pediococcus inopinatus LB-400 strain of the present disclosure (FIG. 7A) and extracellular vesicles (EVs) derived from the LB-400 strain (FIGS. 7B and 7C) by WST assay (FIG. 7A), trypan blue exclusion assay (FIG. 7B) and cell images (FIG. 7C).

FIG. 8 shows a result of investigating the protection of nerves from oxidative stress in neuroblastoma in which oxidative stress-induced cell death is induced by H₂O₂ (hydrogen peroxide) by a Pediococcus inopinatus LB-400 strain of the present disclosure by WST assay at 450 nm.

FIG. 9 shows a result of investigating the protection of nerves in which cell death is induced by Aβ (amyloid beta) accumulation by a culture of a Pediococcus inopinatus LB-400 strain of the present disclosure by WST assay at 450 nm.

FIGS. 10A and 10B show a result of investigating the effect of inhibiting the activity of acetylcholinesterase (AChE) of a culture of a Pediococcus inopinatus LB-400 strain of the present disclosure (FIG. 10A) and EVs derived from the LB-400 strain (FIG. 10B).

FIG. 11 shows a result of investigating the effect of reducing the production of the inflammatory cytokine IL-1ß in microglia (BV2 cells) when EVs derived from a Pediococcus inopinatus LB-400 strain of the present disclosure are treated together with LPS.

FIG. 12 shows a result of investigating the effect of increasing the production of the anti-inflammatory cytokine IL-10 in microglia (BV2 cells) when EVs derived from a Pediococcus inopinatus LB-400 strain of the present disclosure are treated together with LPS.

FIG. 13 shows photographs obtained by observing the time-dependent biodistribution, by IVIS, of extracellular vesicles (ExoPN-101) derived from a Pediococcus inopinatus strain, stained with a near-infrared (NIR) fluorescent dye, in a mouse model after injection.

FIG. 14 shows photographs comparing the expression of extracellular vesicles (ExoPN-101) derived from a Pediococcus inopinatus strain, stained with a near-infrared (NIR) fluorescent dye, in brain tissue, observed by IVIS, in a mouse model administered with the extracellular vesicles, relative to a control group administered with PBS.

FIG. 15 is a confocal microscopic photograph showing the brain distribution of extracellular vesicles (ExoPN-101) derived from a Pediococcus inopinatus strain, stained with a red fluorescent dye, in a mouse model after injection.

FIG. 16 is a photograph showing the protein expression level of alpha-synuclein in the substantia nigra of mouse brain, measured after administering ExoPN-101-ASO, which is extracellular vesicles (ExoPN-101) derived from a Pediococcus inopinatus strain loaded with an antisense oligonucleotide (ASO), to a Parkinson's disease-induced mouse model.

FIG. 17 shows the results of identifying dopaminergic neuronal cell death in the striatum and substantia nigra regions by tyrosine hydroxylase (TH) staining after administering ExoPN-101-ASO, which is extracellular vesicles (ExoPN-101) derived from a Pediococcus inopinatus strain loaded with an antisense oligonucleotide (ASO), to a Parkinson's disease-induced mouse model.

FIG. 18 is a schematic diagram showing the schedule for administering siRNA-loaded extracellular vesicles (siRNA-ExoPN-101) to mice by intracerebroventricular injection (ICV) or intravenous injection (IV).

FIG. 19 is a graph comparing the expression level of the AgRP gene in the hypothalamus of mice administered with siRNA1 or siRNA1-ExoPN-101, as analyzed by real-time polymerase chain reaction (qPCR).

FIG. 20 is a graph comparing the expression level of the NPY gene in the hypothalamus of mice administered with siRNA2 or siRNA2-ExoPN-101, as analyzed by real-time polymerase chain reaction (qPCR).

### BEST MODE

Hereinafter, the present disclosure is described in more detail through examples. These examples are provided only to specifically describe the present disclosure, and it will be obvious to those having ordinary knowledge in the art that the scope of the present disclosure is not limited by the examples.

### EXAMPLES

### 1. Preparation of Strain

A Pediococcus inopinatus LB-400 strain (accession number KCCM12653P, Korean Culture Center of Microorganisms) was inoculated to an MRS broth medium to 1% and cultured for 24 hours. Then, after conducting centrifugation at 3500 rpm for 10 minutes at 4 °C, the remaining MRS broth medium was removed by washing the bacteria with PBS (phosphate-buffered saline). Subsequently, the bacteria homogenized in PBS were prepared at 10-fold (MOI=10), 1-fold (MOI=1) and 0.1-fold (MOI=0.1) for treatment to cells.

### 2. Preparation of Culture of Strain

A Pediococcus inopinatus LB-400 strain was inoculated to an MRS broth medium to 1% and cultured for 24 hours. Then, after conducting centrifugation at 3500 rpm for 10 minutes at 4 °C, the remaining MRS broth medium was removed by washing the bacteria with PBS (phosphate-buffered saline). Subsequently, the bacteria were inoculated to a DMEM (Dulbecco's modified Eagle's medium, Welgene, Korea) at 1×10⁹ CFU/mL and cultured at 30 °C for 24 hours. Then, a culture obtained by centrifuging the same at 8,000 rpm for 5 minutes at 4 °C was adjusted to pH 7.2 and then filtered with a syringe filter (pore size=0.2 μm).

3. Isolation of Extracellular Vesicles (EVs) from Culture of Strain

After fractionating the culture of the Pediococcus inopinatus LB-400 strain to 100 kDa and 300 kDa using a centrifugal filter, samples were obtained by finally treating with a 0.22 μm filter. As a result of comparing the content of extracellular vesicles in the fractions using Nanosight LM 10 (Malvern Panalytical, Korea), the contents of the particles with cutoff values of 0-100, 100-300 and 300 or higher were 35%, 35% and 30%, respectively (4×10⁷ particles/mL).

### 4. SEM Imaging of Extracellular Vesicles

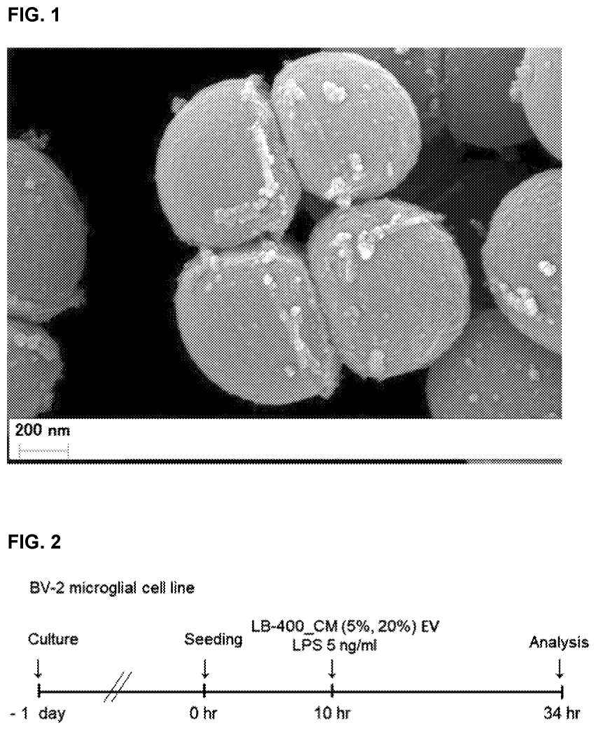

After washing the prepared Pediococcus inopinatus LB-400 strain twice with 0.1 M PB (phosphate buffer) for 30 minutes, followed by post-fixation in OsO₄ for 2 hours, the strain was dehydrated sequentially with 50-100% ethanol. Then, after sufficiently drying using a critical point dryer (Leica EM CPD 300), followed by platinum coating using an ion sputter (Leica EM ACE600), the strain was imaged by field emission scanning electron microscopy (FE-SEM Merlin, Zeiss) (FIG. 1).

### 5. Preparation of Microglia

For testing of anti-neuroinflammatory response, microglia (BV-2 cells) acquired from Ewha Womans University were cultured in DMEM containing 5% FBS (fetal bovine serum) (GIBCO, BRL) and 1% penicillin/streptomycin (Welgene, Korea) in a 5% CO₂ incubator at 37 °C (FIG. 2).

### 6. Preparation of Neuroblastoma

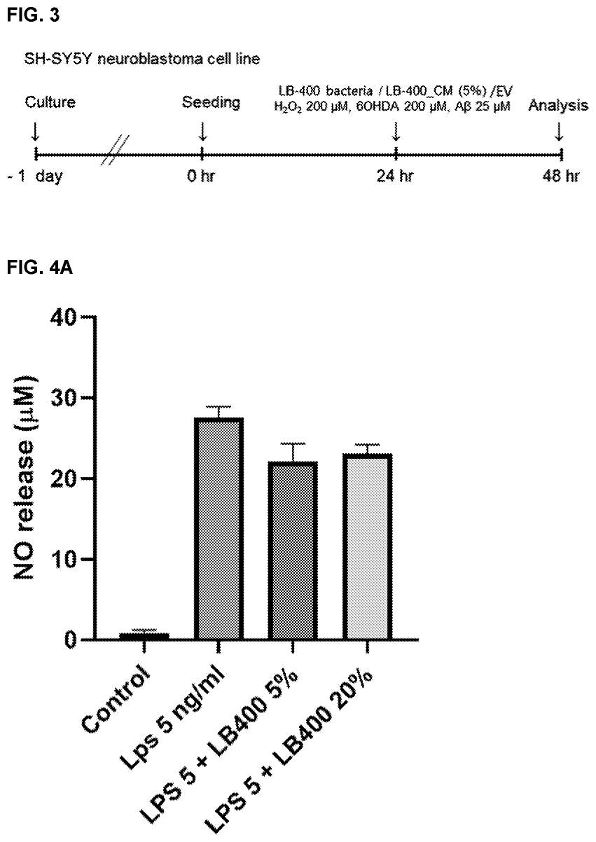

For testing of neuron-protecting effect, neuroblastoma (SH-SY5Y cells, Cat. 22266, Korean Cell Line Bank) were cultured in DMEM/F-12 containing 10% FBS (fetal bovine serum) (GIBCO, BRL) and 1% penicillin/streptomycin (Welgene, Korea) in a 5% CO₂ incubator at 37 °C (FIG. 3).

7. Investigation of Neuroinflammation-Inhibiting Effect of Culture of Pediococcus inopinatus and EVs

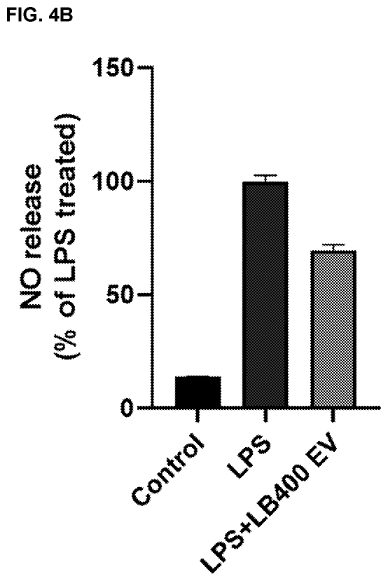

In order to investigate the neuroinflammation-inhibiting effect of the culture of a Pediococcus inopinatus LB-400 strain (FIG. 4a) and extracellular vesicles (EVs) derived from the LB-400 strain (FIG. 4b), BV-2 microglial cells were treated at 2×10⁵ cell/mL. The culture of LB-400 was diluted with a medium to 5% or 20%. LPS (lipopolysaccharide) was diluted with a medium to 5 ng/ml (FIG. 4). EVs were treated at 1×10⁷ particles/mL, and a control group (con) was treated with PBS of the same volume.

8. Investigation of Neuron-Protecting Effect of Pediococcus inopinatus and EVs

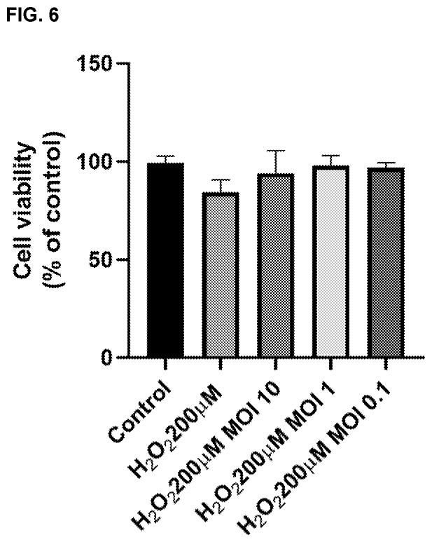

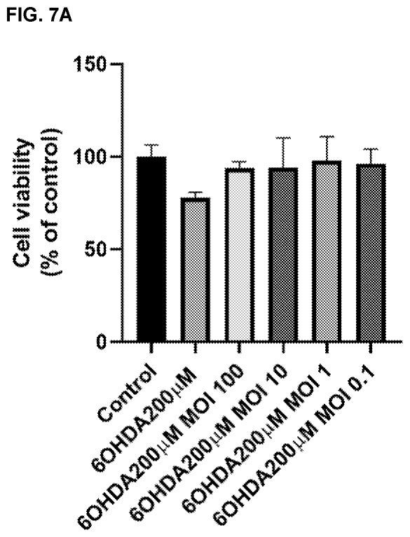

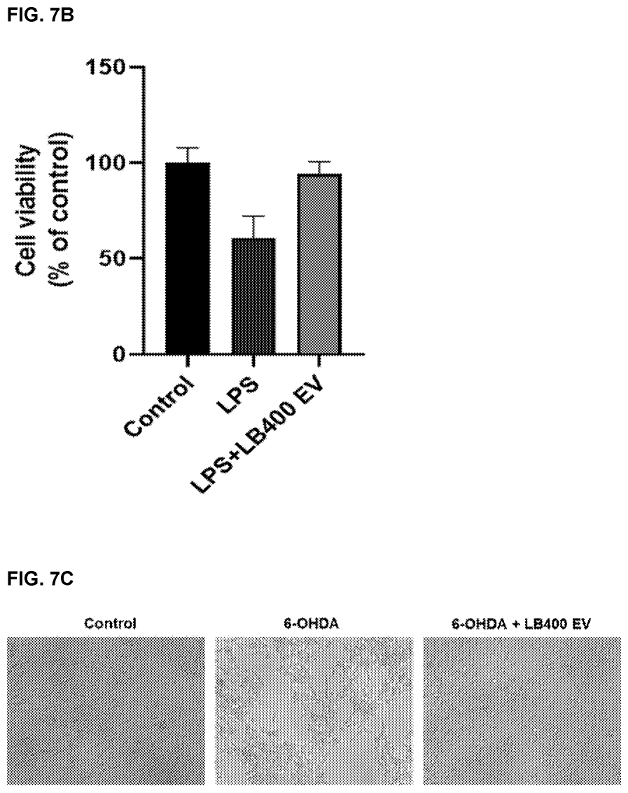

In order to investigate the neuron-protecting effect of the Pediococcus inopinatus LB-400 strain, the LB-400 bacteria were treated with SH-SY5Y neuroblastoma cells at 1×10⁶ cell/mL, at 10-fold (MOI=10), 1-fold (MOI=1) and 0.1-fold (MOI=0.1). EVs were treated at 4×10⁶ particle/mL. H₂O₂ (hydrogen peroxide) (FIG. 6) and 6-OHDA (6-hydroxydopamine) (FIG. 7) were used after diluting in a medium to 200 μM. A control group (con) was treated with PBS of the same volume.

9. Investigation of Neuron-Protecting Effect of Culture of Pediococcus inopinatus

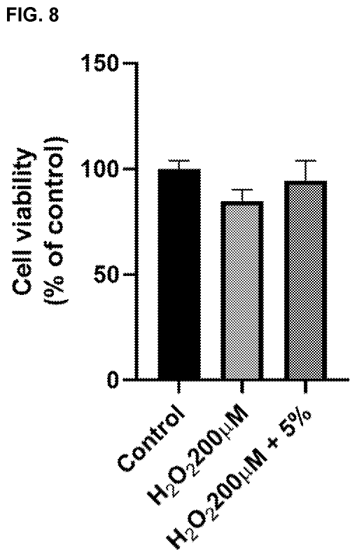

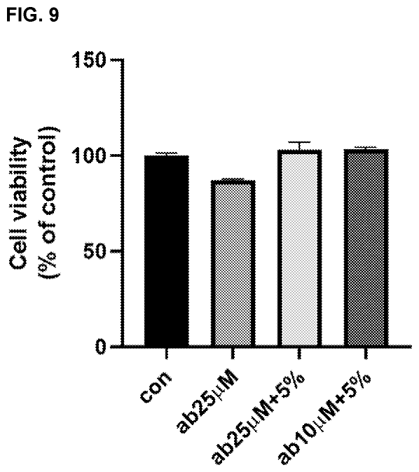

In order to investigate the neuron-protecting effect of the Pediococcus inopinatus LB-400 strain, SH-SY5Y neuroblastoma cells were treated at 1×10⁶ cell/mL. The culture of LB-400 was diluted with a medium to 5%. H₂O₂ (Hydrogen peroxide) (FIG. 8) was used after diluting in a medium to 200 μM, and Aß (amyloid beta) (FIG. 9) used after diluting in a medium to 25 μM.

### 10. NO Assay

After reacting 100 μL of a supernatant with 100 μL of a Griess reagent (1% sulfanilamide, 0.1% N-(1-naphthyl)-ethylenediamine dihydrochloride, 2.5% H₃PO₄), nitric oxide production was measured at 540 nm using a Multiskan sky 96-well microplate spectrophotometer (Thermo Scientific, USA) (FIG. 4).

### 11. WST-8 Cell Viability Assay

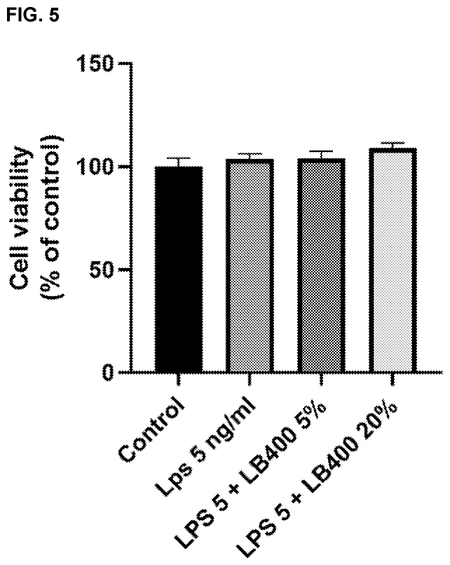

WST-8 assay is a colorimetric method for quantification of living cells. After treating cells with 10 μL of Quanti-Max WST-8 per well and incubating in a CO₂ incubator for 1 hour, cell viability was measured at 450 nm using a Multiskan sky 96-well microplate spectrophotometer (Thermo Scientific, USA) (FIG. 5).

### 12. Trypan Blue Exclusion Assay

Living cells were stained with trypan blue. After treating cells with extracellular vesicles (EVs) derived from the Pediococcus inopinatus LB-400 strain and 6-OHDA, the cell suspension was mixed with trypan blue at 1:1. After conducting reaction for 2 minutes, the number of cells was counted under a phase-contrast microscope. Cell viability was determined by calculating the percentage of unstained cells (FIG. 7b).

13. Investigation of Effect of Inhibiting the Activity of Acetylcholinesterase (AChE) of Culture of Pediococcus inopinatus and EVs

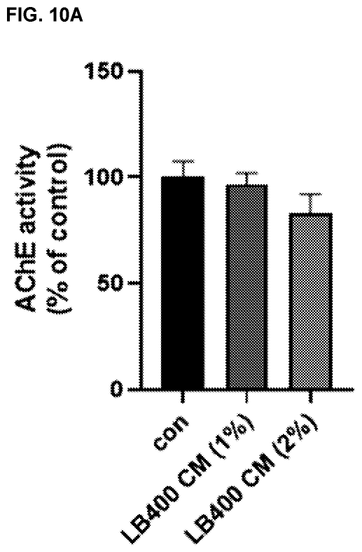

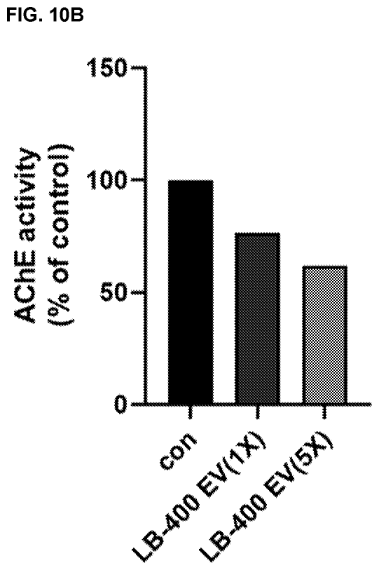

In order to investigate the effect of improving memory or cognitive ability of the culture of the Pediococcus inopinatus LB-400 strain and extracellular vesicles (EVs) derived therefrom, the effect of inhibiting the activity of acetylcholinesterase (AChE) was evaluated (FIG. 10). The inhibitory effect against acetylcholinesterase (AChE) was measured using an AChE activity assay kit (Sigma). After adding AChE (5 units/mL), each sample (LB-400 culture or EVs) and a buffer solution to a 96-well plate, the reaction mixture was reacted at room temperature for 10 minutes. The enzymatic activity of AChE was determined by measuring absorbance at 410 nm using a microplate reader, with respected to a control group (con) not treated with the sample as 100%. The control group (con) was treated with a medium and PBS of the same volume. The LB-400 culture was used after diluting to 1% or 2%, and the EVs were treated at 4×10⁵ particles/mL (1×) or 2×10⁶ particles/mL (5×).

14. Investigation of Effect of Reducing Inflammatory Cytokine Production of EVs Derived from Pediococcus inopinatus

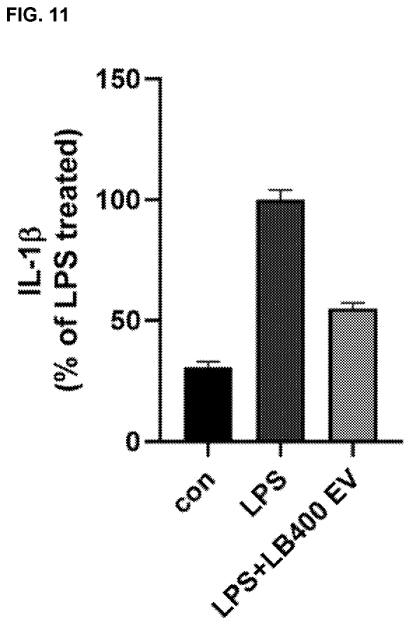

The effect of reducing the production of the inflammatory cytokine IL-1β of extracellular vesicles (EVs) derived from the Pediococcus inopinatus LB-400 strain was investigated (FIG. 11). Experiment was conducted for a cell supernatant obtained by treating cells with LPS and EVs using an IL-1β ELISA kit (Invitrogen). IL-1β production was determined by measuring absorbance at 450 nm using a Multiskan sky microplate spectrophotometer (Thermo Scientific, USA). LPS (lipopolysaccharide) was used after diluting with a medium to 200 ng/ml. The EVs were treated at 1×10⁷ particles/mL, and a control group (con) was treated with PBS of the same volume.

15. Investigation of Effect of Increasing Anti-Inflammatory Cytokine Production of EVs Derived from Pediococcus inopinatus

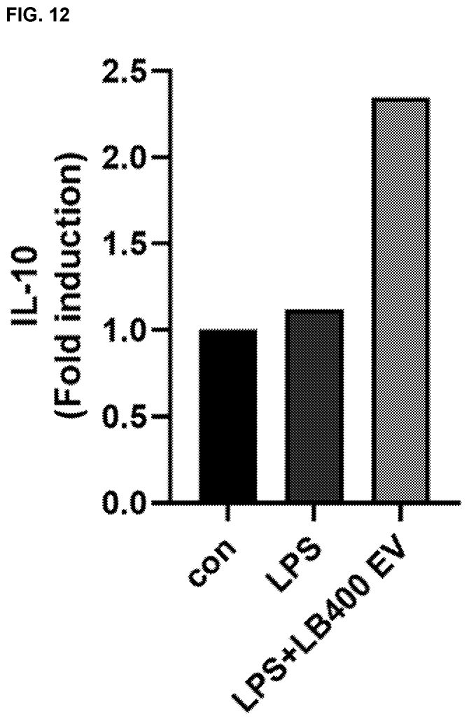

The effect of increasing the production of the anti-inflammatory cytokine IL-10 of extracellular vesicles (EVs) derived from the Pediococcus inopinatus LB-400 strain was investigated (FIG. 12). Experiment was conducted for a cell supernatant obtained by treating cells with LPS and EVs using an IL-10 ELISA kit (MyBioSource). IL-10 production was determined by measuring absorbance at 450 nm using a Multiskan sky microplate spectrophotometer (Thermo Scientific, USA). LPS (lipopolysaccharide) was used after diluting with a medium to 200 ng/ml. The EVs were treated at 1×10⁷ particles/mL, and a control group (con) was treated with PBS of the same volume.

16. Confirmation of Biodistribution of Extracellular Vesicles Derived from Pediococcus inopinatus Strain in Mice

The biodistribution of extracellular vesicles derived from the Pediococcus inopinatus strain was analyzed using mice.

Specifically, ExoPN-101 prepared in Example 3 was stained with an ExoGlow™-Vivo EV Labeling Kit (Near IR) and then administered to the tail vein of mice at a dose of 1×10¹¹ particles/mouse. The biodistribution of the stained ExoPN-101 was measured by IVIS at 2, 6 and 24 hours after administration, and the fluorescence in the brain excised from the mice at 2 hours after administration was also measured by IVIS.

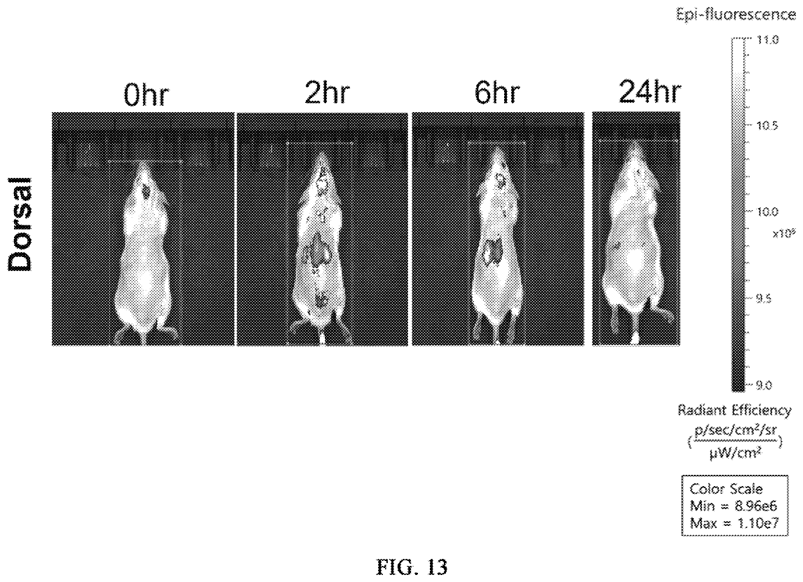

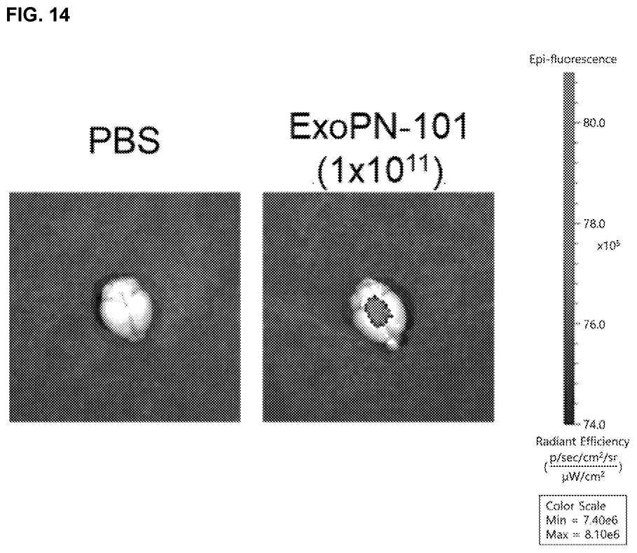

As a result, the stained ExoPN-101 was mainly detected in the brain, liver and bladder (FIG. 13). A strong fluorescence signal in the brain region was detected up to 6 hours, and the fluorescence signal decreased after 24 hours. In particular, the strongest fluorescence signal in the brain was observed at 2 hours after ExoPN-101 administration; accordingly, the mice were sacrificed at 2 hours after administration and the fluorescence intensity in the excised brain was measured, confirming that a strong fluorescence signal was detected in the brain upon administration of the stained ExoPN-101, compared to a control group administered with PBS (FIG. 14). Meanwhile, a fluorescence signal was also observed in the bladder region, confirming that ExoPN-101 can be excreted through urine.

From these results, it was confirmed that extracellular vesicles derived from the Pediococcus inopinatus strain can be delivered to the brain through systemic circulation and can be removed from the body by being excreted through urine.

17. Confirmation of Brain Distribution of Extracellular Vesicles Derived from Pediococcus inopinatus Strain in Mice

The brain distribution of extracellular vesicles derived from the Pediococcus inopinatus strain was analyzed using mice.

Specifically, ExoPN-101 prepared in Example 3 was stained with an ExoGlow™-Protein EV Labeling kit (Red) and then administered to the tail vein of mice at a dose of 1×10¹⁰ particles/mouse. At 2 hours after administration, the mice were sacrificed and the brain was excised, fixed in 4% PFA (paraformaldehyde), sagittally sectioned to a thickness of 20 μm, and observed under a confocal microscope.

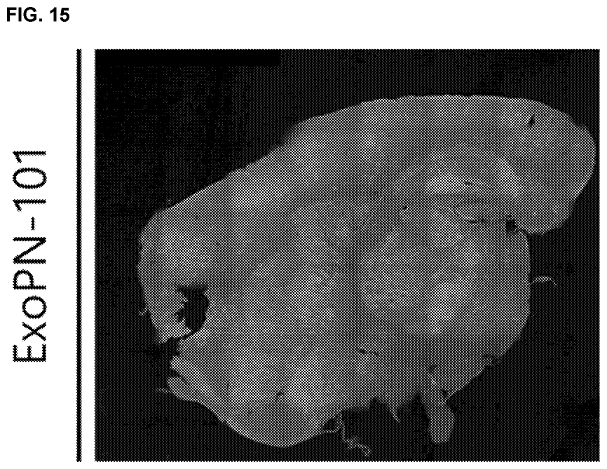

As a result, it was confirmed that ExoPN-101 was evenly distributed throughout the mouse brain tissue, and that the distribution was particularly high in the deep regions of the brain (FIG. 15).

18. Preparation of Extracellular Vesicles Derived from Pediococcus inopinatus Strain Loaded with an Antisense Oligonucleotide

In order to confirm whether extracellular vesicles derived from the Pediococcus inopinatus strain can be utilized as a drug delivery carrier, extracellular vesicles loaded with an antisense oligonucleotide (ASO) were prepared.

Specifically, 200 nM of an RNase H-mediated gapmer ASO directly targeting the mRNA of the SNCA gene, which encodes the alpha-synuclein protein in mice, and 1×10¹⁰ particles of ExoPN-101 were placed in the same tube and incubated at 37 °C for 30 minutes. Then, 167 μL of Exoquick-TC solution was added and the mixture was reacted overnight at 4 °C, followed by centrifugation at 10,000g for 10 minutes to remove the supernatant. The pellet was washed twice with PBS. Finally, the pellet of extracellular vesicles loaded with the ASO (ExoPN-101-ASO) was suspended in PBS according to the amount to be used. The nucleic acid sequences of the ASOs used are shown in Table 1 below.

TABLE 1

| | Nucleic acid sequence | |
|---|---|---|
| Name | (5′→3′) | SEQ ID NO |
| hASO | GTTTTCATCAATATCTGCAA | SEQ ID NO: 1 |
| mASO | TTTAATTACTTCCACCA | SEQ ID NO: 2 |

### 19. Induction of Parkinson's Disease Using MPTP and Probenecid in a Mouse Model and Administration of ExoPN-101-ASO

In order to confirm whether extracellular vesicles derived from the Pediococcus inopinatus strain can be utilized as a drug delivery carrier, extracellular vesicles loaded with an antisense oligonucleotide (ASO) were administered to an animal model in which Parkinson's disease was induced.

Specifically, in order to induce Parkinson's disease in the animal model, MPTP and probenecid were intraperitoneally injected into C57BL6 mice at doses of 25 mg/kg and 250 mg/kg, respectively. Probenecid was injected 30 minutes before MPTP, and the injections were performed twice a week for a total of 10 times to induce Parkinson's disease. Thereafter, ExoPN-101-ASO prepared in Example 18 was administered to the tail vein at a dose of 1×10¹⁰ particles, twice a week for a total of 4 times, starting from the day after the 7th MPTP administration. For comparison of efficacy, the same amount of ASO alone and ExoPN-101 alone were each administered to the tail vein in the same manner as ExoPN-101-ASO, twice a week for a total of 4 times. The mice were sacrificed and the brains were excised 2 weeks after the last administration.

### 20. Confirmation of the Inhibitory Effect on Alpha-Synuclein by Administration of ASO-Loaded Extracellular Vesicles in a Parkinson's Disease-Induced Mouse Model

In order to confirm whether extracellular vesicles derived from the Pediococcus inopinatus strain can be utilized as a drug delivery carrier, the expression of alpha-synuclein in the substantia nigra (SN) of the brain tissue excised in Example 19 was measured.

Specifically, the brain tissue was placed in a solution containing 1% Triton X-100, 2 mM EDTA, 1% protease inhibitor and 1% phosphatase inhibitor and homogenized, followed by sonication and centrifugation to isolate proteins, which were quantified using bovine serum albumin (BSA). Equal amounts of protein were mixed with a sample buffer containing SDS and β-mercaptoethanol, boiled at 98 °C for 10 minutes, and subjected to electrophoresis and transfer. The membrane was blocked with 5% skim milk and reacted overnight at 4 °C with Purified Mouse Anti-α-Synuclein antibody (BD Biosciences) at 1:1,000 dilution and Anti-γ-Tubulin antibody (Sigma) at 1:2,000 dilution, washed three times with Tris-buffered saline with Tween 20 (TBST), and then reacted with a secondary antibody (anti-mouse IgG-HRP) at 1:2,000 dilution for 1 hour at room temperature. The protein bands were treated with an ECL (enhanced chemiluminescence) solution and analyzed using ImageQuant software. The expression level of alpha-synuclein relative to gamma-tubulin was calculated and normalized to the Parkinson's disease-induced group.

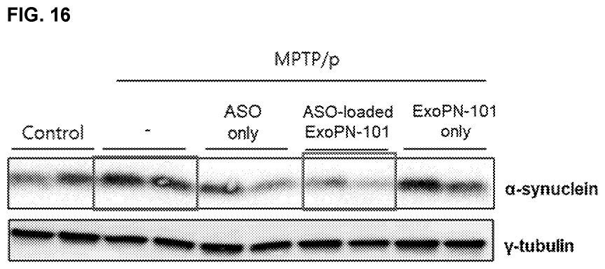

As a result of the protein analysis, the level of alpha-synuclein was significantly reduced in the group administered with ExoPN-101-ASO (FIG. 16). In contrast, the group administered with ASO alone exhibited a lower SNCA expression-inhibiting effect than the group administered with ExoPN-101-ASO, confirming the effect of ExoPN-101 on brain penetration and intracellular uptake of the ASO.

From these results, it was confirmed that ExoPN-101, which is an extracellular vesicle derived from the Pediococcus inopinatus strain, can efficiently deliver an ASO to a target site in the brain, particularly the substantia nigra, thereby inhibiting the expression of SNCA.

### 21. Confirmation of the Improving Effect on Dopaminergic Neurons by Administration of ASO-Loaded Extracellular Vesicles in a Parkinson's Disease-Induced Mouse Model

In order to confirm whether extracellular vesicles derived from the Pediococcus inopinatus strain can be utilized as a drug delivery carrier, dopaminergic neurons in the brain tissue excised in Example 19 were stained and analyzed.

Specifically, the brain tissue was fixed in 4% PFA at 4 °C. Paraffin blocks were prepared from the fixed tissue, and the striatum and substantia nigra regions were each sectioned to a thickness of 5 μm, mounted on slides, and stained with tyrosine hydroxylase (TH), a marker of dopaminergic neurons. The optical density of the stained striatum and substantia nigra regions was then measured and normalized to the Parkinson's disease-induced group.

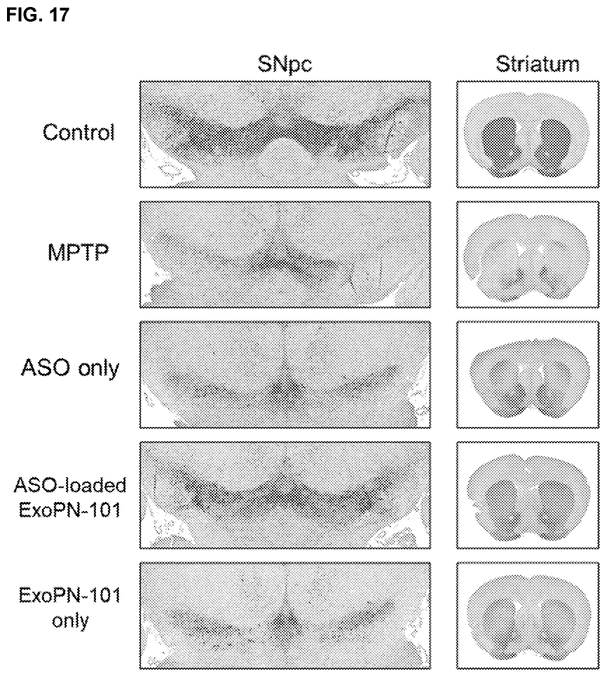

As a result, a significantly higher dopaminergic neuron-preserving effect was observed in the group administered with ExoPN-101-ASO, compared to the groups treated with ExoPN-101 alone or ASO alone (FIG. 17). That is, it was confirmed that administration of ExoPN-101-ASO not only inhibits the accumulation of alpha-synuclein but also enables the inhibition of pathological progression of Parkinson's disease and functional neuronal recovery.

From these results, it was confirmed that ExoPN-101, which is an extracellular vesicle derived from the Pediococcus inopinatus strain, can effectively deliver an ASO loaded therein to deep regions of the brain by mere tail-vein administration in mice, thereby inducing an effect of inhibiting the pathology of a brain disease.

22. Preparation of Extracellular Vesicles Derived from Pediococcus inopinatus Strain Loaded with siRNA

In order to confirm whether extracellular vesicles derived from the Pediococcus inopinatus strain can be utilized as a drug delivery carrier, extracellular vesicles loaded with siRNA were prepared.

Specifically, siRNAs targeting the mRNA of the genes encoding AgRP and NPY, which are appetite-stimulating neuropeptides produced in the mouse hypothalamus, were used (Table 2). siRNA1 or siRNA2 and ExoPN-101 were placed in the same tube and incubated at 37 °C for 30 minutes. The mixture was then centrifuged through a 100 kDa Amicon tube to remove and purify residual siRNA. The amount of each siRNA contained in the finally recovered siRNA1-ExoPN-101 and siRNA2-ExoPN-101 was quantified and used in subsequent experiments according to the amount required.

TABLE 2

| | Target | Nucleic acid sequence | SEQ ID |
|---|---|---|---|
| Name | gene | (5′→3′) | NO |
| SIRNA1 | AgRP | GCACUGCCAUAUAAGCUCA | SEQ ID |
| | | UGAGCUUAUAUGGCAGUGC | NO: 3 |
| SIRNA2 | NPY | CUCAUCACCAGACAGAGAU | SEQ ID |
| | | AUCUCUGUCUGGUGAUGAG | NO: 4 |

23. Evaluation of the Target Gene Expression-Inhibiting Effect after Tail-Vein Administration of siRNA-ExoPN-101 in a Mouse Model

In order to confirm whether extracellular vesicles derived from the Pediococcus inopinatus strain can be utilized as a drug delivery carrier, siRNA-loaded extracellular vesicles were administered to an animal model.

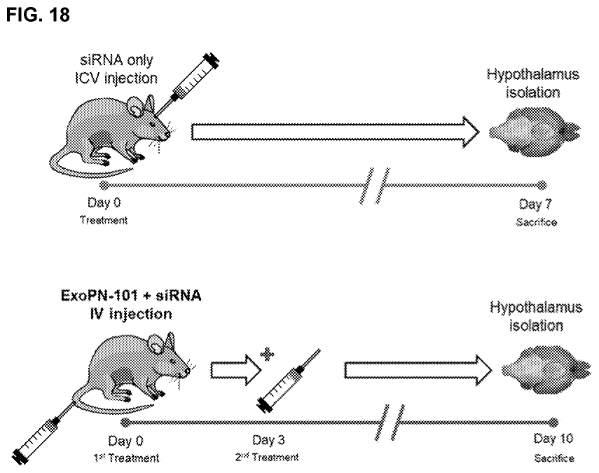

Specifically, C57BL6 mice were divided into a negative control group administered with PBS by intravenous injection, a positive control group administered with siRNA alone by intracerebroventricular injection (ICV), and experimental groups administered with siRNA1-ExoPN-101 or siRNA2-ExoPN-101 by intravenous injection (FIG. 18). The loading amounts of siRNA in siRNA1-ExoPN-101 and siRNA2-ExoPN-101 were 62.4 ng/3×10¹⁰ particles and 75.0 ng/3×10¹⁰ particles, respectively. The dose for tail-vein administration was 3×10¹⁰ particles/100 μL, and the dose for direct intracerebral injection was 5 μg/10 μL.

The negative control group was administered PBS twice by tail-vein injection. For comparison of efficacy, the positive control group was administered the same amount of siRNA1 or siRNA2 as the experimental groups, once by intracerebroventricular injection (ICV) directly into the brain. The experimental groups were administered siRNA1-ExoPN-101 or siRNA2-ExoPN-101, prepared in Example 22, twice by tail-vein injection. One week after the final administration, the mice were sacrificed and the brains were excised. Thereafter, the expression of the genes encoding the hypothalamus-specific neuropeptides AgRP or NPY in the hypothalamus and cortex of the excised brain tissue was measured by qPCR.

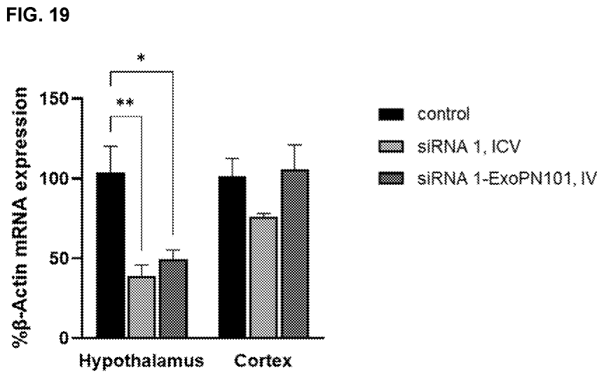

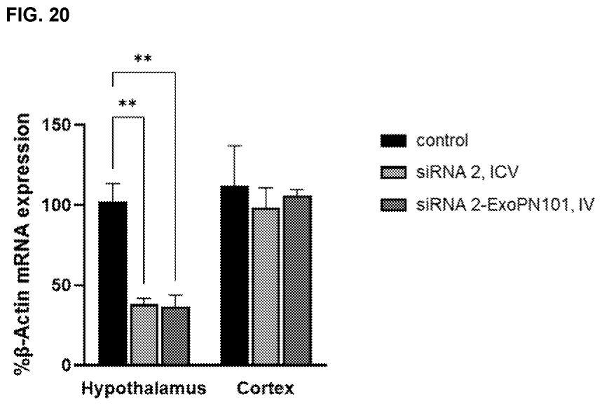

As a result, no significant difference in target gene expression was observed in the cortex between the negative control group and the experimental groups. However, in the hypothalamus, the expression of the target gene was significantly inhibited for both siRNA1 (FIG. 19) and siRNA2 (FIG. 20). In addition, the gene-silencing efficacy achieved by tail-vein administration was confirmed to be at a level similar to that achieved by direct intracerebral administration.

From these results, it was confirmed that ExoPN-101, which is an extracellular vesicle derived from the Pediococcus inopinatus strain, can effectively deliver siRNA loaded therein to deep regions of the brain by mere tail-vein administration in mice, thereby inducing a target gene-silencing effect.

Although the present disclosure was described using the examples, those having ordinary knowledge in the art will be able to change and modify the present disclosure without departing from the technical idea of the present disclosure and such changes or modifications are also included in the scope of the present disclosure.

### [Microorganism Deposit According to Budapest Treat on the International Recognition of the Deposit of Microorganisms for the Purposes of Patent Procedure]

Depository agency: Korean Culture Center of Microorganisms
   Yurim B/D, 45, Hongjenae-2ga-gil, Seodaemun-gu
   Seoul 03641
   Republic of Korea
Accession number: KCCM12653P
Date of deposition: Jan. 14, 2020

1. A method for alleviating or treating a CNS disease in a subject in need thereof, comprising administering a therapeutically effective amount of a composition comprising i) extracellular vesicles derived from Pediococcus inopinatus, a culture, a lysate, an extract or a fermented product thereof, and ii) a drug, to a subject,
wherein the extracellular vesicles cross the blood-brain barrier (BBB) and deliver the drug to the brain of the subject.

2. The method according to claim 1, wherein the CNS disease is one or more disease selected from the group consisting of a cerebrovascular disease, a degenerative brain disease, a neurogenetic disease, an inflammatory brain disease, epilepsy and a CNS tumor.

3. The method according to claim 2, wherein:
(i) the cerebrovascular disease is one or more disease selected from the group consisting of stroke, cerebral infarction, cerebral hemorrhage and cerebral aneurysm;
(ii) the neurogenetic disease is one or more disease selected from the group consisting of Dravet syndrome, SMA (Spinal Muscular Atrophy), Angelman syndrome, Rett syndrome and Spinocerebellar ataxia;
(iii) the inflammatory brain disease is one or more disease selected from the group consisting of Multiple sclerosis, Neuromyelitis optica spectrum disorder (NMOSD);
(iv) the CNS tumor is one or more disease selected from the group consisting of glioblastoma, Astrocytoma, Medulloblastoma, Brain metastases and Spinal cord tumor; or
(v) the degenerative brain disease is one or more disease selected from the group consisting of Parkinson's disease, Alzheimer's disease, Huntington's disease, Lou Gehrig's disease (amyotrophic lateral sclerosis), Creutzfeldt-Jakob disease, stroke, multiple sclerosis, learning disorder, cognitive impairment, neuroinflammation, neuronal damage and memory impairment.

4. The method according to claim 1, wherein the composition is administered via an administration route selected from the group consisting of intravenous administration, intraperitoneal administration, intramuscular administration, subcutaneous administration, intranasal administration, and intradermal administration.

5. The method according to claim 1, wherein the extracellular vesicles are substantially excreted from the body of the subject via urine within 24 hours following administration.

6. The method according to claim 1, wherein the drug is an antisense oligonucleotide (ASO) that targets mRNA encoding alpha-synuclein, and the administering reduces alpha-synuclein protein accumulation in the substantia nigra of the subject.

7. The method according to claim 6, wherein the composition preserves dopaminergic neurons in the striatum and substantia nigra of the subject.

8. The method according to claim 1, wherein the drug is an siRNA that targets mRNA encoding a neuropeptide.

9. The method according to claim 8, wherein the neuropeptide is selected from the group consisting of agouti-related peptide (AgRP) and neuropeptide Y (NPY).

10. A method for delivering a drug across the blood-brain barrier (BBB) into the brain of a subject, comprising administering to the subject a composition comprising extracellular vesicles derived from Pediococcus inopinatus, a culture, a lysate, an extract or a fermented product thereof, and the drug.

11. The method according to claim 10, wherein the composition is administered via an administration route selected from the group consisting of intravenous administration, intraperitoneal administration, intramuscular administration, subcutaneous administration, intranasal administration and intradermal administration.

12. The method according to claim 10, wherein the drug is selected from the group consisting of a nucleic acid, a peptide, a protein, an antibody, a gene editing agent, a vector, a small molecule compound, a radionuclide, an imaging agent, a diagnostic agent, a peptide nucleic acid (PNA), and a combination thereof.

13. The method according to claim 12, wherein the drug is selected from the group consisting of an antisense oligonucleotide (ASO), a small interfering RNA (siRNA), a short hairpin RNA (shRNA), a double-stranded RNA (dsRNA), a messenger RNA (mRNA), a microRNA (miRNA), a single guide RNA (sgRNA) or CRISPR component, a DNA, and an aptamer.

14. The method according to claim 13, wherein:
(a) the drug is an antisense oligonucleotide that targets mRNA encoding alpha-synuclein; or
(b) the drug is an siRNA that targets mRNA encoding a neuropeptide selected from the group consisting of agouti-related peptide (AgRP) and neuropeptide Y (NPY).

15. A composition for crossing the blood-brain barrier (BBB), comprising extracellular vesicles derived from Pediococcus inopinatus, a culture, a lysate, an extract or a fermented product thereof,
wherein the extracellular vesicles are capable of crossing the BBB.

16. The composition according to claim 15, wherein the Pediococcus inopinatus is Pediococcus inopinatus WIKIM27 (accession number KCCM12653P).

17. The composition according to claim 15, wherein the extracellular vesicles have a diameter of 10 nm to 1,000 nm.

18. The composition according to claim 15, the composition further comprises a drug loaded onto or within the extracellular vesicles,
wherein the extracellular vesicles are capable of delivering the drug across the BBB into the brain.

19. A composition for alleviating or treating a CNS disease, comprising:
extracellular vesicles derived from Pediococcus inopinatus, a culture, a lysate, an extract or a fermented product thereof; and
a drug loaded onto or within the extracellular vesicles,
wherein the extracellular vesicles are capable of crossing the blood-brain barrier (BBB) to deliver the drug to the brain.

20. The composition according to claim 19, wherein the Pediococcus inopinatus is Pediococcus inopinatus WIKIM27 (accession number KCCM12653P).

21. The composition according to claim 19, wherein the drug is selected from the group consisting of a nucleic acid, a peptide, a protein, an antibody, a gene editing agent, a vector, a small molecule compound, a radionuclide, an imaging agent, a diagnostic agent, a peptide nucleic acid (PNA), and a combination thereof.

22. The composition according to claim 19, wherein the CNS disease is one or more disease selected from the group consisting of a cerebrovascular disease, a degenerative brain disease, a neurogenetic disease, an inflammatory brain disease, epilepsy and a CNS tumor.

23. The composition according to claim 19, wherein the extracellular vesicles have a diameter of about 10 nm to about 800 nm.

24. The composition according to claim 19, wherein the composition comprises 1×10⁴ to 1×10¹³ of the extracellular vesicles per administration.

25. The composition according to claim 19, wherein the extracellular vesicles comprise one or more selected from the group consisting of exosomes, microvesicles, ectosomes, and apoptotic bodies.

26. A method for preparing a composition for alleviating or treating a CNS disease, comprising:
(a) a step of culturing Pediococcus inopinatus in a culture medium; and
(b) a step of obtaining extracellular vesicles loaded with a drug,
wherein the extracellular vesicles loaded with the drug are capable of crossing the blood-brain barrier (BBB) to deliver the drug to the brain.

27. The method according to claim 26, wherein step (b) comprises:
co-culturing the Pediococcus inopinatus together with the drug such that the drug is incorporated into the extracellular vesicles during the co-culturing; and
isolating the extracellular vesicles loaded with the drug from the culture.

28. The method according to claim 26, wherein step (b) comprises:
isolating extracellular vesicles from the culture of step (a); and
incubating the isolated extracellular vesicles with the drug under conditions sufficient for the drug to be incorporated into the extracellular vesicles, thereby obtaining the extracellular vesicles loaded with the drug.

29. The method according to claim 26, wherein the drug is selected from the group consisting of a nucleic acid, a peptide, a protein, an antibody, a gene editing agent, a vector, a small molecule compound, a radionuclide, an imaging agent, a diagnostic agent, a peptide nucleic acid (PNA), and a combination thereof.

30. The method according to claim 29, wherein:
(a) the drug is an antisense oligonucleotide that targets mRNA encoding alpha-synuclein; or
(b) the drug is an siRNA that targets mRNA encoding a neuropeptide selected from the group consisting of agouti-related peptide (AgRP) and neuropeptide Y (NPY).


SEQUENCE LISTING (4 sequences)

SEQ ID NO: 1 - DNA, 20 bp, synthetic construct
  misc_feature 1..20: note = hASO
  source 1..20: mol_type = other DNA
gttttcatcaatatctgcaa

SEQ ID NO: 2 - DNA, 17 bp, synthetic construct
  misc_feature 1..17: note = mASO
  source 1..17: mol_type = other DNA
tttaattacttccacca

SEQ ID NO: 3 - RNA, 38 nt, synthetic construct
  source 1..38: mol_type = other RNA
  misc_feature 1..38: note = note = siRNA1_AgRP
gcactgccatataagctcatgagcttatatggcagtgc

SEQ ID NO: 4 - RNA, 38 nt, synthetic construct
  source 1..38: mol_type = other RNA
  misc_feature 1..38: note = siRNA2_NPY
ctcatcaccagacagagatatctctgtctggtgatgag